(19) United States
(12) Patent Application Publication
(10) Pub. No.: US 2021/0251200 A1
(43) Pub. Date: Aug. 19, 2021

(54) PRODUCTION METHOD FOR ANIMAL MODELS WITH DISEASE ASSOCIATED PHENOTYPES

(71) Applicant: Recombinetics, Inc., Eagan, MN (US)

(72) Inventors: Daniel F. Carlson, Woodbury, MN (US); Staci Lyn Solin, Minneapolis, MN (US)

(21) Appl. No.: 17/187,121

(22) Filed: Feb. 26, 2021

Related U.S. Application Data
(63) Continuation of application No. PCT/US2019/049231, filed on Aug. 30, 2019.
(60) Provisional application No. 62/725,643, filed on Aug. 31, 2018.

(51) Int. Cl. A01K 67/027; C12N 15/873; C12N 15/90

(52) U.S. Cl. CPC A01K 67/0276; C12N 15/873; A01K 2267/0387; A01K 2217/075; A01K 2227/108; C12N 15/907

(57) ABSTRACT

The present disclosure provides methods to produce non-human animal models for diseases that have a poor life-expectancy. The animal models provided herein are the result of gene editing to result in genetic lesions that recapitulate human diseases by virtue of introgressing lethal, dominant negative or non-functional mutations in animal genomes corresponding to those responsible for human diseases. In some cases, the genomic edit may result in a low number of pregnancies carried to term and/or a failure to thrive phenotype with those born failing to survive to sexual maturity. The present disclosure provides methods to produce non-chimeric animals containing a detrimental genetic lesion from healthy chimeric animals. In this method, the chimeric animals are derived from cells in which the genetic lesion is made with the defect being complemented by the genome of an animal that is gametogenically deficient (cannot produce gametes) and cannot pass on its own genes. Thus, the gametes of the chimera are completely derived from the edited animal. When a male and female chimera are mated with each other, the offspring are 100% of the edited genome.

### CROSS-REFERENCE

This application is a continuation of International Application No. PCT/US2019/049231 filed Aug. 30, 2019, which application claims the benefit of U.S. Provisional Application No. 62/725,643 filed Aug. 31, 2018, all of which are incorporated by reference herein in their entirety.

### STATEMENT AS TO FEDERALLY SPONSORED RESEARCH

This invention was made with the support of the United States government under Contract number R44GM108150 by National Institute of General Medical Sciences of the National Institutes of Health.

### BACKGROUND OF THE DISCLOSURE

Non-rodent preclinical animal models, for example, swine models, are useful for biomedical research because swine and other non-rodent animals can more closely model human disease. Accordingly, there is a growing need for reproductive methods for producing such animals.

### SUMMARY OF THE DISCLOSURE

Currently, somatic cell nuclear transfer (SCNT) is the most frequently used approach for the generation of genetically modified swine models. Although well established, SCNT is hindered by inefficiency and incomplete reprogramming that results in developmental defects and neonatal mortality and is therefore not an effective production method. Propagation by breeding also suffers inefficiencies due to recessive inheritance, segregation of multiple loci and disease severity or lethality requiring heterozygous breeders. Disclosed herein are breeding methods based on Deleted-in-Azoospermia-like (DAZL) knockout (KO) animals (also referred to herein as “DAZL null”). Since the DAZL null animals have an ablated germline, they are the ideal base genetics for germline stem cell transplantation (GST) and blastocyst complementation (BC). This system enables breeding of disease models and lineage or organogenesis-deficient lines that could not otherwise be bred due to high morbidity or mortality.

In some embodiments are animal models that simulate diseases, including for example, dilated cardiomyopathy (DCM) or severe combined immunodeficiency (SCID). The DCM model results in high neonatal morbidity, making it an ideal disease model for propagation using GST approach. The SCID model is generated by multiplex knockout of IL2Rg and RAG2 resulting in complete absence of T- B- and NK cells. SCID pigs cannot easily be reared to breeding age and intercross of heterozygotes is inefficient for production of double null animals. As donors for BC, DAZL null cells can rescue the T-, B- and NK-deficiency phenotype in a host, but do not contribute to the sexually mature germline resulting in gamete production only from the complemented SCID host. As a result, intercrosses between immune-restored chimeras will result in 100% useful T-, B- and NK-deficient offspring.

Therefore, the ability to establish a DAZL platform that enables germline rescue of swine models and production of biomedical swine at a rate that is commercially attractive and feasible for exogenic organ production is needed. Efficient production of valuable failure to thrive and high morbidity gene edited model lines will allow acceleration and development of new therapies, devices and medical treatments for complex diseases in humans.

Disclosed herein are methods to produce non-human animal models for diseases that result in a failure to thrive. The animal models provided herein are the result of gene editing to result in genetic lesions that recapitulate human diseases by virtue of introgressing lethal, dominant negative or non-functional mutations in animal genomes corresponding to those responsible for human diseases. In some cases, the genomic edit may result in a low number of pregnancies carried to term or those born failing to survive to sexual maturity. The present disclosure provides methods to produce non-chimeric animals containing a detrimental genetic lesion from healthy chimeric animals. In this method, the chimeric animals are derived from host embryos in which the genetic lesion is made with the defect being complemented by the genome of a donor cell that is gametogenically deficient (cannot produce gametes) and cannot pass on its own genes. Thus, the gametes of the chimera are completely derived from the edited animal. When a male and female chimera are mated with each other, the offspring are 100% of the edited genome.

Therefore, in one exemplary embodiment, disclosed is a method of producing non-human animal models having congenital defects comprising: i. editing a host cell to create one or more genetic lesions/defects in an animal model; ii. cloning the fibroblast or primary cell to provide a first line; iii. creating an embryo from the cell; iv. complementing the genetic defects in the development of the embryo by providing a donor cell that does not comprise the genetic lesion/defects of the first line with the donor cell being gametogenically deficient. In these and other embodiments the gametogenically deficient cell or animal is a deleted-in-azoospermia-like knockout (DAZL^{-/-}) cell or animal.

In various exemplary embodiments, the method further comprises: v. harvesting germline stem cells (GSC) from the chimera; vi. transplanting the GSC from the chimera into the gonads (testis or ovaries) of a gametogenically deficient animal; wherein the GSC differentiate into sperm or ova; wherein the sperm are used to impregnate a female, chimera or wildtype of step iii; wherein the ova are fertilized by the sperm of a male chimera of claim 1, step iii; wherein the resulting progeny have the genotype of the first/host line and are homozygous for the genetic lesions.

In various other embodiments, the method includes, breeding a female chimera with a male chimera to provide non-chimeric progeny that are solely of the first line/have congenital defects. In some embodiments the animal is a livestock animal. In various embodiments the livestock animal is a pig, goat sheep or cattle. In some embodiments the animal is a mini-pig. In these and other embodiments the lesion is found in, but not limited to one or more of, RNA-Binding Motif Protein 20 (RBM20), Interleukin 2 Receptor Subunit Gamma (IL2Rg), Recombination Activating 2 (RAG2), polycystin-1 (PKD1), polycystin 2 (PKD2), and/or Fibrocystin/Polyductin (PKHD1). In some embodiments the animal is heterozygous for the one or more gene edits. In yet other embodiments the animal is homozygous for the one or more gene edits. In other embodiments, the cell is a primary cell, a fibroblast or a stem cell.

In yet other exemplary embodiments, disclosed is a method of producing a non-human animal model having congenital defects comprising: i) creating one or more genetic lesions or defects in a first cell to provide a genotype of a first line; ii) providing a second cell that is gametogenically deficient and is of a second line; iii) cloning the first and second cells to provide first and second embryos; iv) using the first or second embryos as a host and the remaining embryo as a donor; v) transferring one or more cells from the donor embryo and implanting them in the host embryo to create a healthy chimera by complementation of the genetic defects of the host; vi) wherein the gametes of the chimera have the genotype of the host line; vii) breeding a male and female of the host line to provide offspring that are non-chimeric and only of the host line. In embodiments the donor embryo is of the first line. In yet other embodiments, the donor embryo is of the second line. In these embodiments, those of skill in the art will appreciate that the host embryo is of the different line than the donor. In various embodiments the animal is a livestock animal. In some embodiments the livestock animal is cattle, pig, goat or sheep. In some embodiments the animal is a pig. In various embodiments the pig is a minipig. In various embodiments the gametogenically deficient animal is a Deleted-in-Azoospermia-like knockout (DAZL^{-/-}) animal.

In various embodiments disclosed the genetic lesion comprises one or more genes comprising RNA-Binding Motif Protein 20 (RBM20), Interleukin 2 Receptor Subunit Gamma (IL2Rg), Recombination Activating 2 (RAG2), polycystin-1 (PKD1), polycystin 2 (PKD2), and/or Fibrocystin/Polyductin (PKHD1). In some aspect the animal is heterozygous for one or more genetic lesion. In other aspects the animal is homozygous for one or more lesion. In these and other embodiments the first cell is a fibroblast, primary cell or stem cell. In various embodiments the second cell is a fibroblast, primary cell or stem cell.

Disclosed herein are methods of breeding an animal with a genetic edit that causes a failure to thrive phenotype comprising obtaining a host blastocyst, embryo, or morula from the animal with the genetic edit that causes the failure to thrive phenotype and introducing to the host blastocyst, embryo, or morula, a donor cell from a donor animal that comprises a deleted-in-azoospermia like (DAZL) knock out mutation and does not comprise the genetic edit that causes the failure to thrive phenotype to create a chimeric blastocyst, embryo, or morula. In some embodiments, the failure to thrive phenotype comprises a reduced ability to produce offspring that survive to sexual maturity relative to an animal that does not have the genetic edit that causes the failure to thrive phenotype. In some embodiments, the failure to thrive phenotype comprises a reduced ability to grow or a reduced ability to reach maturity relative to an animal that does not have the genetic edit that causes the failure to thrive phenotype. In some embodiments, the failure to thrive phenotype comprises a lineage deficiency phenotype or an organogenesis deficiency phenotype. In some embodiments, the donor animal does not produce sufficient functional gametes to reproduce. In some embodiments, the chimeric blastocyst, embryo, or morula is implanted into a surrogate mother to produce an offspring of the animal with the genetic edit that causes the failure to thrive phenotype. In some embodiments, the offspring comprises the genetic edit that causes the failure to thrive phenotype. In some embodiments, the offspring is heterozygous for the genetic edit that causes the failure to thrive phenotype. In some embodiments, the offspring is homozygous for the genetic edit that causes the failure to thrive phenotype. In some embodiments, the surrogate mother does not comprise the genetic edit that causes the failure to thrive phenotype. In some embodiments, the offspring does not comprise a genotype of the donor animal. In some embodiments, the genetic edit that causes the failure to thrive phenotype comprises a genetic edit in a gene selected from the group consisting RNA-Binding Motif Protein 20 (RBM20), Interleukin 2 Receptor Subunit Gamma (IL2Rg), Recombination Activating 2 (RAG2), polycystin-1 (PKD1), polycystin 2 (PKD2), and Fibrocystin/Polyductin (PKHD1). In some embodiments, the animal with the genetic edit that causes the failure to thrive phenotype or the donor animal or the animal with the genetic edit that causes the failure to thrive phenotype and the donor animal is a livestock animal. In some embodiments, the livestock animal is selected from the group consisting of cattle, pig, goat, and sheep. In some embodiments, the pig is a mini-pig. In some embodiments, the mini-pig is selected from the group consisting of Ossabaw, Goettingen, Yucatan, Bama Xiang Zhu, Wuzhishan and Xi Shuang Banna. In some embodiments, the donor cell is a stem cell.

Disclosed herein are chimeric blastocysts, embryos, or morulas comprising a host blastocyst, embryo, or morula from an animal with a genetic edit that causes a failure to thrive phenotype and a donor cell from a donor animal with a DAZL knock out mutation and without the genetic edit that causes the failure to thrive phenotype. In some embodiments, the failure to thrive phenotype comprises a reduced ability to produce offspring that survive to sexual maturity relative to an animal that does not have the genetic edit that causes the failure to thrive phenotype. In some embodiments, the failure to thrive phenotype comprises a reduced ability to grow or a reduced ability to reach maturity relative to an animal that does not have the genetic edit that causes the failure to thrive phenotype. In some embodiments, the failure to thrive phenotype comprises a lineage deficiency phenotype or an organogenesis deficiency phenotype. In some embodiments, the donor animal does not produce sufficient functional gametes to reproduce. In some embodiments, the genetic edit that causes the failure to thrive phenotype comprises a genetic edit in a gene selected from the group consisting RNA-Binding Motif Protein 20 (RBM20), Interleukin 2 Receptor Subunit Gamma (IL2Rg), Recombination Activating 2 (RAG2), polycystin-1 (PKD1), polycystin 2 (PKD2), and Fibrocystin/Polyductin (PKHD1). In some embodiments, the animal with the genetic edit that causes the failure to thrive phenotype or the donor animal or the animal with the genetic edit that causes the failure to thrive phenotype and the donor animal is a livestock animal. In some embodiments, the livestock animal is selected from the group consisting of cattle, pig, goat, and sheep. In some embodiments, the pig is a mini-pig. In some embodiments, the mini-pig is selected from the group consisting of Ossabaw, Goettingen, Yucatan, Bama Xiang Zhu, Wuzhishan and Xi Shuang Banna. In some embodiments, the donor cell is a stem cell.

Disclosed herein are surrogate mothers comprising an implanted chimeric blastocyst, embryo, or morula wherein the chimeric blastocyst, embryo, or morula comprises a host blastocyst, embryo, or morula from an animal with a genetic edit that causes a failure to thrive phenotype and a donor cell from a donor animal with a deleted-in-azoospermia like (DAZL) knock out mutation and without the mutation that causes the failure to thrive phenotype. In some embodiments, the failure to thrive phenotype comprises a reduced ability to produce offspring that survive to sexual maturity relative to an animal that does not have the genetic edit that causes the failure to thrive phenotype. In some embodiments, the failure to thrive phenotype comprises a reduced ability to grow or a reduced ability to reach maturity relative to an animal that does not have the genetic edit that causes the failure to thrive phenotype. In some embodiments, the failure to thrive phenotype comprises a lineage deficiency phenotype or an organogenesis deficiency phenotype. In some embodiments, the donor animal does not produce sufficient functional gametes to reproduce. In some embodiments, the genetic edit that causes the failure to thrive phenotype comprises a genetic edit in a gene selected from the group consisting RNA-Binding Motif Protein 20 (RBM20), Interleukin 2 Receptor Subunit Gamma (IL2Rg), Recombination Activating 2 (RAG2), polycystin-1 (PKD1), polycystin 2 (PKD2), and Fibrocystin/Polyductin (PKHD1). In some embodiments, the animal with the genetic edit that causes the failure to thrive phenotype or the donor animal or the animal with the genetic edit that causes the failure to thrive phenotype and the donor animal is a livestock animal. In some embodiments, the livestock animal is selected from the group consisting of cattle, pig, goat, and sheep. In some embodiments, the pig is a mini-pig. In some embodiments, the mini-pig is selected from the group consisting of Ossabaw, Goettingen, Yucatan, Bama Xiang Zhu, Wuzhishan and Xi Shuang Banna. In some embodiments, the donor cell is a stem cell. In some embodiments, the surrogate mother is a livestock animal. In some embodiments, the livestock animal is selected from the group consisting of cattle, pig, goat, and sheep. In some embodiments, the pig is a mini-pig. In some embodiments, the mini-pig is selected from the group consisting of Ossabaw, Goettingen, Yucatan, Bama Xiang Zhu, Wuzhishan and Xi Shuang Banna. In some embodiments, the surrogate mother does not comprise the genetic edit that causes the failure to thrive phenotype. Disclosed herein are the animals produced from the implanted chimeric blastocyst, embryo, or morula of the above embodiments. Disclosed herein are the progeny of the animals of the previous embodiment.

Disclosed herein are methods of breeding an animal with a genetic edit that causes a failure to thrive phenotype comprising introducing a germline stem cell from the animal with the genetic edit that causes the failure to thrive phenotype to a testis of a host animal that comprises a deleted-in-azoospermia like (DAZL) knock out mutation and that does not comprise the genetic edit that causes the failure to thrive phenotype wherein the germline stem cell introduced to the testis matures to produce a functional sperm comprising the genetic edit that causes the failure to thrive phenotype. In some embodiments, the failure to thrive phenotype comprises a reduced ability to produce offspring that survive to sexual maturity relative to an animal that does not have the genetic edit that causes the failure to thrive phenotype. In some embodiments, the failure to thrive phenotype comprises a reduced ability to grow or a reduced ability to reach maturity relative to an animal that does not have the genetic edit that causes the failure to thrive phenotype. In some embodiments, the failure to thrive phenotype comprises a lineage deficiency phenotype or an organogenesis deficiency phenotype. In some embodiments, the functional sperm comprising the genetic edit that causes the failure to thrive phenotype is used to fertilize a donor ovum to produce an embryo. In some embodiments, the donor ovum is heterozygous for the genetic edit that causes the failure to thrive phenotype. In some embodiments, the donor ovum does not comprise the genetic edit that causes the failure to thrive phenotype. In some embodiments, the embryo is implanted into a surrogate mother to produce an offspring comprising the genetic edit that causes the failure to thrive phenotype. In some embodiments, the offspring is heterozygous for the genetic edit that causes the failure to thrive phenotype. In some embodiments, the offspring is homozygous for the genetic edit that causes the failure to thrive phenotype. In some embodiments, the offspring does not comprise a genotype of the host animal that comprises the DAZL knock out mutation. In some embodiments, the genetic edit that causes the failure to thrive phenotype comprises a genetic edit in a gene selected from the group consisting RNA-Binding Motif Protein 20 (RBM20), Interleukin 2 Receptor Subunit Gamma (IL2Rg), Recombination Activating 2 (RAG2), polycystin-1 (PKD1), polycystin 2 (PKD2), and Fibrocystin/Polyductin (PKHD1). In some embodiments, the animal with the genetic edit that causes the failure to thrive phenotype is a livestock animal. In some embodiments, the livestock animal is selected from the group consisting of cattle, pig, goat, and sheep. In some embodiments, the pig is a mini-pig. In some embodiments, the mini-pig is selected from the group consisting of Ossabaw, Goettingen, Yucatan, Bama Xiang Zhu, Wuzhishan and Xi Shuang Banna. In some embodiments, the host animal that comprises the DAZL knock mutation is a livestock animal. In some embodiments, the livestock animal is selected from the group consisting of cattle, pig, goat, and sheep. In some embodiments, the pig is a mini-pig. In some embodiments, the mini-pig is selected from the group consisting of Ossabaw, Goettingen, Yucatan, Bama Xiang Zhu, Wuzhishan and Xi Shuang Banna. In some embodiments, the donor ovum is from an animal that is a livestock animal. In some embodiments, the livestock animal is selected from the group consisting of cattle, pig, goat, and sheep. In some embodiments, the pig is a mini-pig. In some embodiments, the mini-pig is selected from the group consisting of Ossabaw, Goettingen, Yucatan, Bama Xiang Zhu, Wuzhishan and Xi Shuang Banna. In some embodiments, the surrogate mother is a livestock animal. In some embodiments, the livestock animal is selected from the group consisting of cattle, pig, goat, and sheep. In some embodiments, the pig is a mini-pig. In some embodiments, the mini-pig is selected from the group consisting of Ossabaw, Goettingen, Yucatan, Bama Xiang Zhu, Wuzhishan and Xi Shuang Banna.

Disclosed herein are host animals for breeding an animal with a genetic edit that causes a failure to thrive, the host animal comprising a genome with a deleted-in-azoospermia like (DAZL) knock out mutation and that does not comprise the genetic edit that causes the failure to thrive mutation and wherein the host animal comprises a testis containing a transplanted germline stem cell from an animal with the genetic edit that causes the failure to thrive phenotype. In some embodiments, the failure to thrive phenotype comprises a reduced ability to produce offspring that survive to sexual maturity relative to an animal that does not have the genetic edit that causes the failure to thrive phenotype. In some embodiments, the failure to thrive phenotype comprises a reduced ability to grow or a reduced ability to reach maturity relative to an animal that does not have the genetic edit that causes the failure to thrive phenotype. In some embodiments, the failure to thrive phenotype comprises a lineage deficiency phenotype or an organogenesis deficiency phenotype. In some embodiments, the germline stem cell matures to produce a functional sperm comprising the genetic edit that causes the failure to thrive phenotype. In some embodiments, the genetic edit that causes the failure to thrive phenotype comprises a genetic edit in a gene selected from the group consisting RNA-Binding Motif Protein 20 (RBM20), Interleukin 2 Receptor Subunit Gamma (IL2Rg), Recombination Activating 2 (RAG2), polycystin-1 (PKD1), polycystin 2 (PKD2), and Fibrocystin/Polyductin (PKHD1). In some embodiments, the host animal is a livestock animal. In some embodiments, the livestock animal is selected from the group consisting of cattle, pig, goat, and sheep. In some embodiments, the pig is a mini-pig. In some embodiments, the mini-pig is selected from the group consisting of Ossabaw, Goettingen, Yucatan, Bama Xiang Zhu, Wuzhishan and Xi Shuang Banna. In some embodiments, the animal with the genetic edit that causes the failure to thrive phenotype is a livestock animal. In some embodiments, the livestock animal is selected from the group consisting of cattle, pig, goat, and sheep. In some embodiments, the pig is a mini-pig. In some embodiments, the mini-pig is selected from the group consisting of Ossabaw, Goettingen, Yucatan, Bama Xiang Zhu, Wuzhishan and Xi Shuang Banna.

Those of skill in the art will appreciate that the donor cell and the surrogate mother need not be of the same variety or breed. For example, the donor cell may be of a miniature variety while the surrogate may be or a regular or large size. Similarly, the donor cell may be or a medium or large variety animal while the surrogate mother may be a small or medium variety. Indeed, it can be appreciated that the host cell and the donor cell may not be of the same variety, breed or species in order to complement the niche created by the editing of genes.

These and other features and advantages of the present disclosure will be set forth or will become more fully apparent in the description that follows and in the appended claims. The features and advantages may be realized and obtained by means of the instruments and combinations particularly pointed out in the appended claims.

### BRIEF DESCRIPTION OF THE DRAWINGS

The novel features of the disclosure are set forth with particularity in the appended claims. A better understanding of the features and advantages of the disclosure will be obtained by reference to the following detailed description that sets forth illustrative embodiments, in which the principles of the disclosure are utilized, and the accompanying drawings of which:

FIG. 1 is an exemplary schematic of germline stem cell (GSCs) transplantation for propagation of disease models. Using germline stem cell transplantation (GST), alleles of disease model animals where disease phenotype interferes with reproduction are transmitted to offspring. Endogenous GSCs of DAZL null recipients are absent resulting in transmission of exclusively donor genetics.

FIG. 2 is an exemplary schematic of blastocyst complementation for phenotypic rescue. Host (organogenesis-deficient) and donor (DAZL null) embryos are reconstructed by SCNT. Blastomeres from the donor embryo are injected into the host. Successfully complemented chimeric pigs develop into fertile adults. After mating chimeric males and females, the organogenesis-deficient phenotype is transmitted to 100% of offspring.

FIG. 3A-FIG. 3D illustrate characterization of adult DAZL−/− porcine testes. FIG. 3A and FIG. 3B illustrate histology showing the complete absence of germ cells in DAZL−/− adult testes. The basement membrane is highlighted with a dotted line. FIG. 3C illustrates wildtype single or paired spermatogonia (arrows) expressing the marker UCH-L1 are restricted to localization at the basement membrane. FIG. 3D illustrates that UCH-L1 labeling was not detected in adult DAZL−/− testes supporting an absence of spermatogonia.

FIG. 4A-FIG. 4D illustrate immunohistochemical characterization of juvenile DAZL−/− porcine testes. UCH-L1 is a marker for undifferentiated, type A spermatogonia. FIG. 4A illustrates in 10 wk old wildtype testes UCH-L1 positive spermatogonia (arrows) are in contact with somatic cells to form a single layer surrounding the lumen of the tubules. FIG. 4B illustrates UCH-L1 labeling was not detected in 10 wk DAZL−/− testes suggesting an absence of spermatogonia. The basement membrane is highlighted with a dotted line. FIG. 4C and FIG. 4D illustrate expression of the Sertoli cell marker, vimentin, is similar between the 10 wk wildtype and DAZL−/− testes.

FIG. 5A-FIG. 5F illustrate proliferation of porcine germ cells (*) after 1 day (FIG. 5A, FIG. 5C, FIG. 5E, FIG. 5F) and after 7 days culture in vitro (FIG. 5B, FIG. 5D, FIG. 5E, FIG. 5F). Note appearance of cell clusters after 7 days of culture in StemPro medium with addition of growth factors (FIG. 5B and FIG. 5D). Evaluation of EdU incorporation (FIG. 5C, FIG. 5D, FIG. 5F) indicates an increase in proliferation of UCH-L1+ spermatogonia after 7 days of culture in StemPro medium with addition of GDNF, GFRa1, and EGF growth factors (FIG. 5F). UCH-L1 (green), EdU (red), DAPI (blue). Bars=100 μm. For FIG. 5E and FIG. 5F, n=3 experiments each, different letters between bars indicate statistical significance (P<0.05). For FIG. 5E and FIG. 5F, for each plot pair, the 1 day culture plot is on the left-hand side, and the 7 day culture plot is on the right-hand side.

FIG. 6A-FIG. 6E illustrate porcine RBM20 null phenotype characterization. FIG. 6A illustrates Kaplan-Meier survival analysis for RBM20 heterozygous and homozygous R636S mutation demonstrates a strong dose dependent genotype/phenotype correlation with RBM20 mutations. Homozygous animals (bottom line) have a ˜25% survival at 12-weeks with the majority of mortality occurring with sudden neonatal death. FIG. 6B and FIG. 6C illustrate gross pathological samples at 8 weeks of age (LV: left ventricle). FIG. 6D and FIG. 6E illustrate Masons Trichrome staining reveals significant fibrosis in mutant (FIG. 6E) versus control (FIG. 6D).

FIG. 7A-FIG. 7F illustrate restoration of T-, B- and NK-cells in RG-KO (SCID) chimeras. Single cell suspensions were isolated from newborn RG-KO and chimeric RG-KO founders and analyzed by FACS for cell surface markers indicative of T cells (CD3+, CD2+), B cells (CD73a+, CD21+) and NK cells (CD16+, CD2+). T- B- and NK-cells are absent in newborn RG-KO's (FIG. 7A-FIG. 7C) whereas they are restored in chimeric RG-KO founders (FIG. 7D-FIG. 7F).

FIG. 8A-FIG. 8B illustrate micro ovaries in DAZL null females. FIG. 8A illustrates H & E stained cross-section of micro ovary from a 1-year old DAZL null female. Note the absence of follicles in the entire section whereas wild type would have dozens of follicles at this age (wild type not shown). FIG. 8B illustrates the same ovary at 4×.

FIG. 9A- FIG. 9B illustrate successful application of germline stem cell transplantation using genetically similar and divergent breed GSC donors. GSCs isolated from 9 week old Large White (FIG. 9A) or 2 Ossabaw (FIG. 9B) donors were transplanted to one testis of individual 13 week old DAZL KO recipients. Beginning at 6 months of age (sexual maturity) GST recipients were trained for semen collection. Ejaculates were analyzed for the presence of sperm (black arrows) and differentially extracted to reduce the recipient's non-sperm cells within the seminal plasma and concentrate the sperm heads (scale bar 50 um). Single nucleotide polymorphisms (SNP) identified for the recipient tail and donor GSC genomic DNA were PCR amplified and Sanger sequenced. SNP analysis showed transmission of donor-derived sperm in the ejaculates of GST DAZL KO recipients transplanted with Large White (FIG. 9A) or Ossabaw (FIG. 9B) GSCs.

### DETAILED DESCRIPTION OF THE EXEMPLARY EMBODIMENTS

Animal models are important in biomedical research for the study of human diseases poorly recapitulated by rodent species, for the development and testing of preclinical therapeutics in humanized disease models, and as potential sources of xenogeneic or allogeneic organs and tissues. However, the creation and propagation of biomedical swine is plagued by inefficiencies related to animal development, reproduction and lethal phenotypes. Production breeding programs are inadequate when animal models have severe disease-associated phenotypes that reduce long-term viability or the ability to sexually reproduce. For regenerative medicine purposes, the development and propagation of organogenesis-deficient animals also requires an alternative to standard breeding. Disclosed herein are breeding methods which include DAZL null animals and germline stem cell transplantation (GST) and blastocyst complementation (BC) in swine to rescue the germline of valuable lines and followed by propagation of congenital disease and organogenesis-deficient alleles.

Disclosed herein are methods for GST in DAZL null boars culminating with germline transplantation from severe models of dilated cardiomyopathy (DCM) that are inefficiently produced by standard breeding. Disclosed herein are methods including blastocyst complementation for phenotypic rescue of engineered immunodeficient swine as a scale up production method of the immunodeficient model.

### Certain Definitions

Unless defined otherwise, all technical and scientific terms used herein have the same meanings as commonly understood by one of ordinary skill in the art to which this disclosure belongs. All publications and patents specifically mentioned herein are incorporated by reference for all purposes including describing and disclosing the chemicals, instruments, statistical analyses and methodologies which are reported in the publications which might be used in connection with the disclosure. All references cited in this specification are to be taken as indicative of the level of skill in the art. Nothing herein is to be construed as an admission that the disclosure is not entitled to antedate such disclosure by virtue of prior invention.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

“Allele” as used herein refers to an alternate form of a gene. It also can be thought of as variations of DNA sequence. For instance, if an animal has the genotype for a specific gene of Bb, then both B and b are alleles.

References in the specification to “one embodiment”, “an embodiment”, “exemplary embodiment” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The term “and/or” means any one of the items, any combination of the items, or all of the items with which this term is associated.

The phrase “one or more” is readily understood by one of skill in the art, particularly when read in context of its usage. For example, one or more substituents on a phenyl ring refers to one to five, or one to four, for example if the phenyl ring is disubstituted.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of”

As used herein, the terms “including”, “includes”, “having”, “has”, “with”, or variants thereof, are intended to be inclusive similar to the term “comprising.”

“DNA Marker” refers to a specific DNA variation that can be tested for association with a physical characteristic.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term “about” can also modify the end-points of a recited range.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

As used herein, “chimeric” or “chimera” refers to two or more cells in which at least one of the cells is from another animal or another animal embryo, or derived from a cell that is from another animal or another animal embryo. The animal can be of the same or a different species.

“Genome” refers to the genetic makeup of an animal that is the total complement of DNA in its chromosomes.

“Genotype” refers to a particular sequence and a particular allele or loci.

“Genotyping (DNA marker testing)” refers to the process by which an animal is tested to determine the particular alleles it is carrying for a specific genetic test. Organisms may be genotyped to identify various genetic markers. Genetic markers can be a sequence comprising a plurality of bases, or a single nucleotide polymorphism (SNP) at a known location.

“Complex allele” refers to coding region that has more than one mutation within it. This makes it more difficult to determine the effect of a given mutation because researchers cannot be sure which mutation within the allele is causing the effect.

“Homozygous” refers to having two copies of the same allele for a single gene such as BB.

“Heterozygous” refers to having different copies of alleles for a single gene such as Bb.”

“Locus” (plural “loci”) refers to the specific locations of a marker or a gene.

“Chromosomal crossover” (“crossing over”) is the exchange of genetic material between homologous chromosomes inherited by an individual from its mother and father. Each individual has a diploid set (two homologous chromosomes, e.g., 2n) one each inherited from its mother and father. During meiosis I the chromosomes duplicate (4n) and crossover between homologous regions of chromosomes received from the mother and father may occur resulting in new sets of genetic information within each chromosome. Meiosis I is followed by two phases of cell division resulting in four haploid (1n) gametes each carrying a unique set of genetic information. Because genetic recombination results in new gene sequences or combinations of genes, diversity is increased. Crossover usually occurs when homologous regions on homologous chromosomes break and then reconnect to the other chromosome.

“Nucleotide” refers to a structural component of DNA that includes one of the four base chemicals: adenine (A), thymine (T), guanine (G), and cytosine (C).

“Phenotype” refers to the outward appearance of an animal that can be measured. Phenotypes are influenced by the genetic makeup of an animal and the environment.

“Line” as used herein refers to the ancestry or lineage of an animal, especially livestock animals.

“Single Nucleotide Polymorphism (SNP)” is a single nucleotide change in a DNA sequence.

“Haploid genotype” or “haplotype” refers to a combination of alleles, loci or DNA polymorphisms that are linked so as to co-segregate in a significant proportion of gametes during meiosis. The alleles of a haplotype may be in linkage disequilibrium (LD).

The term “restriction fragment length polymorphism” or “RFLP” refers to any one of different DNA fragment lengths produced by restriction digestion of genomic DNA or DNA amplicon with one or more endonuclease enzymes, wherein the fragment length varies between individuals in a population.

“Introgression” also known as “introgressive hybridization”, is the movement of a gene or allele (gene flow) from one species into the gene pool of another by the repeated backcrossing of an interspecific hybrid with one of its parent species. Purposeful introgression is a long-term process; it may take many hybrid generations before the backcrossing occurs.

“Nonmeiotic introgression” genetic introgression via introduction of a gene or allele in a diploid (non-gametic) cell. Non-meiotic introgression does not rely on sexual reproduction and does not require backcrossing and, significantly, is carried out in a single generation. In non-meiotic introgression an allele is introduced into a haplotype via homologous recombination. The allele may be introduced at the site of an existing allele to be edited from the genome or the allele can be introduced at any other desirable site.

As used herein, the term “germ cell deficient” refers to animals that cannot produce germ cells. In cases where animals cannot produce germ cells, they consequently cannot produce gametes, such animals are referred to as “gametogenically deficient” A gametogenically deficient animal cannot pass on its genome sexually, i.e. they cannot contribute to the germline. Those of skill in the art will appreciate that in some instances, an animal may be gametogenically deficient when there is no germ cell deficiency such as when a hormone is lacking that is important in germ cell development to a gamete.

As used herein the term “organogenesis-deficient” animal means an animal whose genome has been modified such that target genes are ablated or modified (a genetic lesion) creating a non-functional gene or gene with altered function. Thus, an ablated/altered gene's ability to provide instructions for organ, cell or tissue development is absent. The combination of one or more ablated genes critical to the development of a particular organ, cell or tissue may create a “niche” for complementation by homologous “donor” genes (cells) from a different genome.

As used herein the term “genetic modification” refers to the direct manipulation of an organiSM'S genome using biotechnology. The term “genetic lesion” refers to the modification of or editing of a gene to be defective or altered in function. The lesion may result in the gene being non-functional, partially functional, or a dominant negative. In some cases, the lesion may be lethal or confer a failure to thrive phenotype.

As used herein the phrase “gene editing”, “genome editing” and “genetic engineering” are synonymous and refer to a process of gene engineering or modification in which DNA is inserted, deleted, modified or replaced in the genome of a living organism. The common methods for such editing use engineered nucleases, or “molecular scissors”. These nucleases create site-specific double-strand breaks (DSBs) at desired locations in the genome. The induced double-strand breaks are repaired through nonhomologous end-joining (NHEJ), microhomology-mediated end joining (MMEJ), single strand annealing (SSA) or homologous recombination (HR), resulting in targeted mutations (‘edits’). Gene editing, the ability to make highly specific changes in the DNA sequence of a living organism, essentially customizing its genetic makeup. Gene editing is performed using nucleases that have been engineered to target a specific DNA sequence, where they introduce cuts into the DNA strands, enabling the removal of existing DNA and the insertion of replacement DNA. Thus, the process of gene editing results in the modification of a specific genomic sequence with no off-target changes or modifications.

As of 2015 four families of engineered nucleases have been used in disrupting DNA: meganucleases, zinc finger nucleases (ZFNs), transcription activator-like effector-based nucleases (TALEN), and the clustered regularly interspaced short palindromic repeats (CRISPR/Cas9) system.

“Transcription activator-like effector nucleases (TALEN5)” one technology for gene editing are artificial restriction enzymes generated by fusing a TAL effector DNA-binding domain to a DNA cleavage domain.

“Zinc finger nucleases (ZFNs)” as used herein are another technology useful for gene editing and are a class of engineered DNA-binding proteins that facilitate targeted editing of the genome by creating double-strand breaks in DNA at user-specified locations.

“Meganuclease” as used herein are another technology useful for gene editing and are endodeoxyribonucleases characterized by a large recognition site (double-stranded DNA sequences of 12 to 40 base pairs); as a result, this site generally occurs only once in any given genome. For example, the 18-base pair sequence recognized by the I-SceI meganuclease would on average require a genome twenty times the size of the human genome to be found once by chance (although sequences with a single mismatch occur about three times per human-sized genome). Meganucleases are therefore considered to be the most specific naturally occurring restriction enzymes.

“CRISPR/CAS” technology as used herein refers to “CRISPRs” (clustered regularly interspaced short palindromic repeats), segments of prokaryotic DNA containing short repetitions of base sequences. Each repetition is followed by short segments of “spacer DNA” from previous exposures to a bacterial virus or plasmid. “CAS” (CRISPR associated protein 9) is an RNA-guided DNA endonuclease enzyme associated with the CRISPR. By delivering the Cas9 protein or RNA and appropriate guide RNAs into a cell, the organism's genome can be cut at any desired location.

“Base Editing” Base editing is a form of genome editing that enables direct, irreversible conversion of one base pair to another at a target genomic locus without requiring double-stranded DNA breaks (DSBs), homology-directed repair (HDR) processes, or donor DNA templates.

Homology directed repair (HDR) is a mechanism in cells to repair ssDNA and double stranded DNA (dsDNA) lesions. This repair mechanism can be used by the cell when there is an HDR template present that has a sequence with significant homology to the lesion site. Specific binding, as that term is commonly used in the biological arts, refers to a molecule that binds to a target with a relatively high affinity compared to non-target tissues, and generally involves a plurality of non-covalent interactions, such as electrostatic interactions, van der Waals interactions, hydrogen bonding, and the like. Specific hybridization is a form of specific binding between nucleic acids that have complementary sequences. Proteins can also specifically bind to DNA, for instance, in TALENs or CRISPR/Cas9 systems or by Gal4 motifs. Introgression of an allele refers to a process of copying an exogenous allele over an endogenous allele with a template-guided process. The endogenous allele might actually be excised and replaced by an exogenous nucleic acid allele in some situations, but present theory is that the process is a copying mechanism. Since alleles are gene pairs, there is significant homology between them. The allele might be a gene that encodes a protein, or it could have other functions such as encoding a bioactive RNA chain or providing a site for receiving a regulatory protein or RNA.

The HDR template is a nucleic acid that comprises a portion of an allele that is being introgressed, an exogenous sequence introduced into the genome or deletion of a portion of an allele. The template may be a dsDNA or a single-stranded DNA (ssDNA). ssDNA templates are preferably from about 20 to about 5000 residues although other lengths can be used. Artisans will immediately appreciate that all ranges and values within the explicitly stated range are contemplated; e.g., from 500 to 1500 residues, from 20 to 100 residues, and so forth. The template may further comprise flanking sequences that provide homology to DNA adjacent to the endogenous allele or the DNA that is to be replaced. Such flanking residues are termed “homology arms” and comprise from 5 to 10 to 40 and up to 200 and 500 bp or more on either side (e.g., “left” and “right” “homology arms”) of the introgressed sequence. Artisans will immediately appreciate that all ranges and values within the explicitly stated range are contemplated. In those cases where a simple deletion is made, the HDR template may simply comprise a homologous sequence reading on either side of the deletion sequence. The template may also comprise a sequence that is bound to a targeted nuclease system and is thus the cognate binding site for the system's DNA-binding member. The term cognate refers to two biomolecules that typically interact, for example, a receptor and its ligand. In the context of HDR processes, one of the biomolecules may be designed with a sequence to bind with an intended, i.e., cognate, DNA site or protein site.

“Indel” as used herein is shorthand for “insertion” or “deletion” referring to a modification of the DNA in an organism.

As used herein the term “renucleated egg” refers to an enucleated egg used for somatic cell nuclear transfer in which the modified nucleus of a somatic cell has been introduced.

“Genetic marker” as used herein refers to a gene/allele or known DNA sequence with a known location on a chromosome. The markers may be any genetic marker e.g., one or more alleles, haplotypes, haplogroups, loci, quantitative trait loci, or DNA polymorphisms [restriction fragment length polymorphisms (RFLPs), amplified fragment length polymorphisms (AFLPs), single nuclear polymorphisms (SNPs), indels, short tandem repeats (STRs), microsatellites and minisatellites]. Conveniently, the markers are SNPs or STRs such as microsatellites, and more preferably SNPs. Preferably, the markers within each chromosome segment are in linkage disequilibrium.

“Blastocyst complementation” as used herein refers to the ability of a cell, generally an embryonic stem cell which retains pluripotency to contribute to a gene edited embryo the missing genetic information (the niche).

As used herein, “native haplotype” or “native genome” means the natural DNA of a particular species or breed of animal that is chosen to be the recipient of a gene or allele that is not present in the host animal.

As used herein the to n “cloning” means production of genetically identical organisms asexually.

“Somatic cell nuclear transfer” (“SCNT”) is one strategy for cloning a viable embryo from a body cell and an egg cell. The technique consists of taking an enucleated oocyte (egg cell) and implanting a donor nucleus from a somatic (body) cell.

### Targeted Endonuclease Systems

Genome editing tools such as transcription activator-like effector nucleases (TALEN5) and zinc finger nucleases (ZFNs) have impacted the fields of biotechnology, gene therapy and functional genomic studies in many organisms. More recently, RNA-guided endonucleases (RGENs) are directed to their target sites by a complementary RNA molecule. The CRISPR/Cas9/CRISPR system is a REGEN. tracrRNA is another such tool that provides specificity to RGENs. These are examples of targeted nuclease systems: these systems have a DNA-binding member that localizes the nuclease to a target site. The site is then cut by the nuclease. TALENs and ZFNs have the nuclease fused to the DNA-binding member. CRISPR/Cas9/CRISPR are cognates that find each other on the target DNA. The DNA-binding member has a cognate sequence in the chromosomal DNA. The DNA-binding member is typically designed in light of the intended cognate sequence so as to obtain a nucleolytic action at or near an intended site. Certain embodiments are applicable to all such systems without limitation; including, embodiments that minimize nuclease re-cleavage, embodiments for making SNPs with precision at an intended residue, and placement of the allele that is being introgressed at the DNA-binding site.

### TALENs

The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA or a TALEN-pair.

The cipher for TALEs has been reported (PCT Publication WO 2011/072246) wherein each DNA binding repeat is responsible for recognizing one base pair in the target DNA sequence. The residues may be assembled to target a DNA sequence. In brief, a target site for binding of a TALEN is determined and a fusion molecule comprising a nuclease and a series of Repeat Variable Diresidues (RVDs) that recognize the target site is created. Upon binding, the nuclease cleaves the DNA so that cellular repair machinery can operate to make a genetic modification near the cut ends. The term TALEN means a protein comprising a Transcription Activator-like (TAL) effector binding domain and a nuclease domain and includes monomeric TALENs that are functional per se as well as others that require dimerization with the nuclease domain of another monomeric TALEN. The dimerization can result in a homodimeric TALEN when both monomeric TALEN are identical or can result in a heterodimeric TALEN when monomeric TALEN are different. TALENs have been shown to induce gene modification in immortalized human cells by means of the two-major eukaryotic DNA repair pathways, non-homologous end joining (NHEJ) and homology directed repair. TALENs are often used in pairs but monomeric TALENs are known. Cells for treatment by TALENs (and other genetic tools) include a cultured cell, an immortalized cell, a primary cell, a primary somatic cell, a zygote, a germ cell, a primordial germ cell, a blastocyst, or a stem cell. In some embodiments, a TAL effector can be used to target other protein domains (e.g., non-nuclease protein domains) to specific nucleotide sequences. For example, a TAL effector can be linked to a protein domain from, without limitation, a DNA 20 interacting enzyme (e.g., a methylase, a topoisomerase, an integrase, a transposase, or a ligase), a transcription activators or repressor, or a protein that interacts with or modifies other proteins such as histones. Applications of such TAL effector fusions include, for example, creating or modifying epigenetic regulatory elements, making site-specific insertions, deletions, or repairs in DNA, controlling gene expression, and modifying chromatin structure.

The term “nuclease” includes exonucleases and endonucleases. The term “endonuclease” refers to any wild-type or variant enzyme capable of catalyzing the hydrolysis (cleavage) of bonds between nucleic acids within a DNA or RNA molecule, preferably a DNA molecule. Non-limiting examples of endonucleases include type II restriction endonucleases such as FokI, HhaI, HindIII, NotI, BbvCI, EcoRI, BgIII, and AlwI. Endonucleases also comprise rare-cutting endonucleases having typically a polynucleotide recognition site of about 12-45 base pairs (bp) in length, more preferably of 14-45 bp. Rare-cutting endonucleases induce DNA double-strand breaks (DSBs) at a defined locus. Rare-cutting endonucleases can for example be a targeted endonuclease, a chimeric Zinc-Finger nuclease (ZFN) resulting from the fusion of engineered zinc-finger domains with the catalytic domain of a restriction enzyme such as FokI or a chemical endonuclease. In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences. Such chemical endonucleases are comprised in the term “endonuclease” according to the present disclosure. Examples of such endonuclease include I-See I, I-Chu L I-Cre I, I-Csm I, PI-See L PI-Tti L PI-Mtu I, I-Ceu I, I-See IL I-See III, HO, PI-Civ I, PI-Ctr L PI-Aae I, PI-Bsu I, PI-Dha I, PI-Dra L PI-May L PI-Meh I, PI-Mfu L PI-Ml I, PI-Mga L PI-Mgo I, PI-Min L PI-Mka L PI-Mle I, PI-Mma I, PI-30 Msh L PI-Msm I, PI-Mth I, PI-Mtu I, PI-Mxe PI-Npu I, PI-Pfu L PI-Rma I, PI-Spb I, PI-Ssp L PI-Fae L PI-Mja I, PI-Pho L PI-Tag L PI-Thy I, PI-Tko I, PI-Tsp I, I-MsoI.

A genetic modification made by nucleases may be, for example, chosen from the list consisting of an insertion, a deletion, insertion of an exogenous nucleic acid fragment, and a substitution. The term insertion is used broadly to mean either literal insertion into the chromosome or use of the exogenous sequence as a template for repair. In general, a target DNA site is identified, and a TALEN-pair is created that will specifically bind to the site. The TALEN is delivered to the cell or embryo, e.g., as a protein, mRNA or by a vector that encodes the TALEN. The TALEN cleaves the DNA to make a double-strand break that is then repaired, often resulting in the creation of an indel, or incorporating sequences or polymorphisms contained in an accompanying exogenous nucleic acid that is either inserted into the chromosome or serves as a template for repair of the break with a modified sequence. This template-driven repair is a useful process for changing a chromosome and provides for effective changes to cellular chromosomes.

The term exogenous nucleic acid means a nucleic acid that is added to the cell or embryo, regardless of whether the nucleic acid is the same or distinct from nucleic acid sequences naturally in the cell. The term nucleic acid fragment is broad and includes a chromosome, expression cassette, gene, DNA, RNA, mRNA, or portion thereof. The cell or embryo may be, for instance, chosen from the group consisting non-human vertebrates, non-human primates, cattle, horse, swine, sheep, chicken, avian, rabbit, goats, dog, cat, laboratory animal, and fish.

Some embodiments involve a composition or a method of making a genetically modified livestock and/or artiodactyl comprising introducing a TALEN-pair into livestock and/or an artiodactyl cell or embryo that makes a genetic modification to DNA of the cell or embryo at a site that is specifically bound by the TALEN-pair and producing the livestock animal/artiodactyl from the cell. Direct injection may be used for the cell or embryo, e.g., into a zygote, blastocyst, or embryo. Alternatively, the TALEN and/or other factors may be introduced into a cell using any of many known techniques for introduction of proteins, RNA, mRNA, DNA, or vectors. Genetically modified animals may be made from the embryos or cells according to known processes, e.g., implantation of the embryo into a gestational host, or various cloning methods. The phrase “a genetic modification to DNA of the cell at a site that is specifically bound by the TALEN”, or the like, means that the genetic modification is made at the site cut by the nuclease domain of the TALEN when the TALEN is specifically bound to its target site. The nuclease does not cut exactly where the TALEN-pair binds, but rather at a defined site between the two binding sites.

Some embodiments involve a composition or a treatment of a cell that is used for cloning the animal. The cell may be a livestock and/or artiodactyl cell, a cultured cell, a primary cell, a primary somatic cell, a zygote, a germ cell, a primordial germ cell, or a stem cell. For example, an embodiment is a composition or a method of creating a genetic modification comprising exposing a plurality of primary cells in a culture to TALEN proteins or a nucleic acid encoding a TALEN or TALENs. The TALENs may be introduced as proteins or as nucleic acid fragments, e.g., encoded by mRNA or a DNA sequence in a vector.

### Zinc Finger Nucleases

Zinc-finger nucleases (ZFNs) are artificial restriction enzymes generated by fusing a zinc finger DNA-binding domain to a DNA-cleavage domain. Zinc finger domains can be engineered to target desired DNA sequences, and this enables zinc-finger nucleases to target unique sequences within complex genomes. By taking advantage of endogenous DNA repair machinery, these reagents can be used to alter the genomes of higher organisms. ZFNs may be used as a method of inactivating genes.

A zinc finger DNA-binding domain has about 30 amino acids and folds into a stable structure. Each finger primarily binds to a triplet within the DNA substrate. Amino acid residues at key positions contribute to most of the sequence-specific interactions with the DNA site. These amino acids can be changed while maintaining the remaining amino acids to preserve the necessary structure. Binding to longer DNA sequences is achieved by linking several domains in tandem. Other functionalities like non-specific FokI cleavage domain (N), transcription activator domains (A), transcription repressor domains (R) and methylases (M) can be fused to a zinc finger protein (ZFP) to form ZFNs respectively, zinc finger transcription activators (ZFA), zinc finger transcription repressors (ZFR, and zinc finger methylases (ZFM) respectively. Materials and methods for using zinc fingers and zinc finger nucleases for making genetically modified animals are disclosed in, e.g., U.S. Pat. No. 8,106,255; U.S. 2012/0192298; U.S. 2011/0023159; and U.S. 2011/0281306.

### Vectors and Nucleic Acids

A variety of nucleic acids may be introduced into cells, for knockout purposes, for inactivation of a gene, to obtain expression of a gene, or for other purposes. As used herein, the term nucleic acid includes DNA, RNA, and nucleic acid analogs, and nucleic acids that are double-stranded or single-stranded (i.e., a sense or an anti sense single strand). Nucleic acid analogs can be modified at the base moiety, sugar moiety, or phosphate backbone to improve, for example, stability, hybridization, or solubility of the nucleic acid. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained.

The target nucleic acid sequence can be operably linked to a regulatory region such as a promoter. Regulatory regions can be porcine regulatory regions or can be from other species. As used herein, operably linked refers to positioning of a regulatory region relative to a nucleic acid sequence in such a way as to permit or facilitate transcription of the target nucleic acid.

In general, any type of promoter can be operably linked to a target nucleic acid sequence. Examples of promoters include, without limitation, tissue-specific promoters, constitutive promoters, inducible promoters, and promoters responsive or unresponsive to a particular stimulus. In some embodiments, a promoter that facilitates the expression of a nucleic acid molecule without significant tissue- or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. In some embodiments, a fusion of the chicken beta actin gene promoter and the CMV enhancer is used as a promoter. See, for example, Xu et al., Hum. Gene Ther. 12:563, 2001; and Kiwaki et al., Hum. Gene Ther. 7:821, 1996.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

A nucleic acid construct may be used that encodes signal peptides or selectable expressed markers. Signal peptides can be used such that an encoded polypeptide is directed to a particular cellular location (e.g., the cell surface). Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphotransferase, thymidine kinase (TK), and xanthine-guanine phosphoribosyl transferase (XGPRT). Such markers are useful for selecting stable transformants in culture. Other selectable markers include fluorescent polypeptides, such as green fluorescent protein or yellow fluorescent protein.

In some embodiments, a sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban et al., Proc. Natl. Acad. Sci., 89:6861, 1992, for a review of Cre/lox technology, and Brand and Dymecki, Dev. Cell, 6:7, 2004. A transposon containing a Cre- or Flp-activatable transgene interrupted by a selectable marker gene also can be used to obtain transgenic animals with conditional expression of a transgene. For example, a promoter driving expression of the marker/transgene can be either ubiquitous or tissue-specific, which would result in the ubiquitous or tissue-specific expression of the marker in FO animals (e.g., pigs). Tissue specific activation of the transgene can be accomplished, for example, by crossing a pig that ubiquitously expresses a marker-interrupted transgene to a pig expressing Cre or Flp in a tissue-specific manner, or by crossing a pig that expresses a marker-interrupted transgene in a tissue-specific manner to a pig that ubiquitously expresses Cre or Flp recombinase. Controlled expression of the transgene or controlled excision of the marker allows expression of the transgene.

In some embodiments, the exogenous nucleic acid encodes a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S-transferase (GST) and FLAG™ tag (Kodak, New Haven, Conn.).

Nucleic acid constructs can be introduced into embryonic, fetal, or adult artiodactyl/livestock cells of any type, including, for example, germ cells such as an oocyte or an egg, a progenitor cell, an adult or embryonic stem cell, a primordial germ cell, a kidney cell such as a PK-15 cell, an islet cell, a beta cell, a liver cell, or a fibroblast such as a dermal fibroblast, using a variety of techniques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells.

In transposon systems, the transcriptional unit of a nucleic acid construct, i.e., the regulatory region operably linked to an exogenous nucleic acid sequence, is flanked by an inverted repeat of a transposon. Several transposon systems, including, for example, Sleeping Beauty (see, U.S. Pat. No. 6,613,752 and U.S. 2005/0003542); Frog Prince (Miskey et al., Nucleic Acids Res., 31:6873, 2003); Tol2 (Kawakami, Genome Biology, 8(Supp.1):S7, 2007); Minos (Pavlopoulos et al., Genome Biology, 8(Suppl.1):52, 2007); Hsmarl (Miskey et al., Mol Cell Biol., 27:4589, 2007); and Passport have been developed to introduce nucleic acids into cells, including mice, human, and pig cells. The Sleeping Beauty transposon is particularly useful. A transposase can be delivered as a protein, encoded on the same nucleic acid construct as the exogenous nucleic acid, can be introduced on a separate nucleic acid construct, or provided as an mRNA (e.g., an in vitro-transcribed and capped mRNA).

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in animals have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, and also any necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., modified HIV-1, SIV or FIV), retrovirus (e.g., ASV, ALV or MoMLV), and transposons (e.g., Sleeping Beauty, P-elements, Tol-2, Frog Prince, piggyBac).

As used herein, the term nucleic acid refers to both RNA and DNA, including, for example, cDNA, genomic DNA, synthetic (e.g., chemically synthesized) DNA, as well as naturally occurring and chemically modified nucleic acids, e.g., synthetic bases or alternative backbones. A nucleic acid molecule can be double-stranded or single-stranded (i.e., a sense or an antisense single strand). The term transgenic is used broadly herein and refers to a genetically modified organism or genetically engineered organism whose genetic material has been altered using genetic engineering techniques. A knockout artiodactyl is thus transgenic regardless of whether or not exogenous genes or nucleic acids are expressed in the animal or its progeny.

### Genetically Modified Animals

Animals may be modified using nucleases or other genetic engineering tools, including recombinase fusion proteins, or various vectors that are known. A genetic modification made by such tools may comprise disruption of a gene. The term disruption of a gene refers to preventing the formation of a functional gene product. A gene product is functional only if it fulfills its normal (wild-type) functions. Disruption of the gene prevents expression of a functional factor encoded by the gene and comprises an insertion, deletion, or substitution of one or more bases in a sequence encoded by the gene and/or a promoter and/or an operator that is necessary for expression of the gene in the animal. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene. Materials and methods of genetically modifying animals are further detailed in U.S. Pat. No. 8,518,701; U.S. 2010/0251395; and U.S. 2012/0222143 which are hereby incorporated herein by reference for all purposes; in case of conflict, the instant specification is controlling. The term trans-acting refers to processes acting on a target gene from a different molecule (i.e., intermolecular). A trans-acting element is usually a DNA sequence that contains a gene. This gene codes for a protein (or microRNA, non-coding RNA or other diffusible molecule) that is used in the regulation the target gene. The trans-acting gene may be on the same chromosome as the target gene, but the activity is via the intermediary protein or RNA that it encodes. Embodiments of trans-acting gene are, e.g., genes that encode targeting endonucleases. Inactivation of a gene using a dominant negative generally involves a trans-acting element. The term cis-regulatory or cis-acting means an action without coding for protein or RNA; in the context of gene inactivation, this generally means inactivation of the coding portion of a gene, or a promoter and/or operator that is necessary for expression of the functional gene.

Various techniques known in the art can be used to inactivate genes to make knock-out animals and/or to introduce nucleic acid constructs into animals to produce founder animals and to make animal lines, in which the knockout or nucleic acid construct is integrated into the genome. Such techniques include, without limitation, pronuclear microinjection (U.S. Pat. No. 4,873,191), retrovirus mediated gene transfer into germ lines (Van der Putten et al., Proc. Natl. Acad. Sci. USA, 82:6148-6152, 1985), gene targeting into embryonic stem cells (Thompson et al., Cell, 56:313-321, 1989), electroporation of embryos (Lo, Mol. Cell. Biol., 3:1803-1814, 1983), sperm-mediated gene transfer (Lavitrano et al., Proc. Natl. Acad. Sci. USA, 99:14230-14235, 2002; Lavitrano et al., Reprod. Fert. Develop., 18:19-23, 2006), and in vitro transformation of somatic cells, such as cumulus or mammary cells, or adult, fetal, or embryonic stem cells, followed by nuclear transplantation (Wilmut et al., Nature, 385:810-813, 1997; and Wakayama et al., Nature, 394:369-374, 1998). Pronuclear microinjection, sperm mediated gene transfer, and somatic cell nuclear transfer are particularly useful techniques. An animal that is genomically modified is an animal wherein all of its cells have the genetic modification, including its germ line cells. When methods are used that produce an animal that is mosaic in its genetic modification, the animals may be inbred and progeny that are genomically modified may be selected. A mosaic animal may be made if some but not all modified (host) cells are complemented (by donor cells) at the blastocyst (multicellular) stage. Animals that are modified so they do not sexually mature can be homozygous or heterozygous for the modification, depending on the specific approach that is used. If a particular gene is inactivated by a knock out modification, homozygosity would normally be required. If a particular gene is inactivated by an RNA interference or dominant negative strategy, then heterozygosity is often adequate.

Typically, in pronuclear or cytoplasmic microinjection, a nucleic acid construct is introduced into a fertilized egg; 1 or 2 cell fertilized eggs are used as the pronuclei containing the genetic material from the sperm head and the egg are visible within the protoplasm. Pronuclear staged fertilized eggs can be obtained in vitro or in vivo (i.e., surgically recovered from the oviduct of donor animals). In vitro fertilized eggs can be produced as follows. For example, swine ovaries can be collected at an abattoir, and maintained at 22-28° C. during transport. Ovaries can be washed and isolated for follicular aspiration, and follicles ranging from 4-8 mm can be aspirated into 50 mL conical centrifuge tubes using 18-gauge needles and under vacuum. Follicular fluid and aspirated oocytes can be rinsed through pre-filters with commercial TL-HEPES (Minitube, Verona, Wis.). Oocytes surrounded by a compact cumulus mass can be selected and placed into TCM-199 OOCYTE MATURATION MEDIUM (Minitube, Verona, Wis.) supplemented with 0.1 mg/mL cysteine, 10 ng/mL epidermal growth factor, 10% porcine follicular fluid, 50 μM 2-mercaptoethanol, 0.5 mg/ml cAMP, 10 IU/mL each of pregnant mare serum gonadotropin (PMSG) and human chorionic gonadotropin (hCG) for approximately 22 hours in humidified air at 38.7° C. and 5% CO₂. Subsequently, the oocytes can be moved to fresh TCM-199 maturation medium, which will not contain cAMP, PMSG or hCG and incubated for an additional 22 hours. Matured oocytes can be stripped of their cumulus cells by vortexing in 0.1% hyaluronidase for 1 minute.

For swine, mature oocytes can be fertilized in 500 μl Minitube PORCPRO IVF MEDIUM SYSTEM (Minitube, Verona, Wis.) in Minitube 5-well fertilization dishes. In preparation for in vitro fertilization (IVF), freshly-collected or frozen boar semen can be washed and resuspended in PORCPRO IVF Medium to 4×10⁵ sperm. Sperm concentrations can be analyzed by computer assisted semen analysis (SPERMVISION, Minitube, Verona, Wis.). Final in vitro insemination can be performed in a 10 μl volume at a final concentration of approximately 40 motile sperm/oocyte, depending on boar. Incubate all fertilizing oocytes at 38.7° C. in 5.0% CO₂ atmosphere for 6 hours. Six hours post-insemination, presumptive zygotes can be washed twice in NCSU-23 and moved to 0.5 mL of the same medium. This system can produce 20-30% blastocysts routinely across most boars with a 10-30% polyspermic insemination rate.

Linearized nucleic acid constructs, mRNAs, ssDNAs or proteins can be injected into one of the pronuclei or cytoplasm. Then the injected eggs can be transferred to a recipient female (e.g., into the oviducts of a recipient female) and allowed to develop in the recipient female to produce the transgenic animals. In particular, in vitro fertilized embryos can be centrifuged at 15,000×g for 5 minutes to sediment lipids allowing visualization of the pronucleus. The embryos can be injected using an Eppendorf FEMTOJET injector and can be cultured until blastocyst formation. Rates of embryo cleavage and blastocyst formation and quality can be recorded.

Embryos can be surgically transferred into uteri of asynchronous recipients. Typically, 20-200 (e.g., 150-200) embryos can be deposited into the ampulla-isthmus junction of the oviduct using a 5.5-inch TOMCAT® catheter. After surgery, real-time ultrasound examination of pregnancy can be performed.

In somatic cell nuclear transfer, a transgenic artiodactyl cell (e.g., a transgenic pig cell or bovine cell) such as an embryonic blastomere, fetal fibroblast, adult ear fibroblast, or granulosa cell that includes a nucleic acid construct or gene modification described above, can be introduced into an enucleated oocyte to establish a combined cell. Oocytes can be enucleated by partial zona dissection near the polar body and then pressing out cytoplasm at the dissection area. Conversely, the cytoplasm can be ejected leaving the nucleus. Typically, an injection pipette with a sharp beveled tip is used to inject the transgenic cell into an enucleated oocyte arrested at meiosis 2. In some conventions, oocytes arrested at meiosis-2 are termed eggs. After producing a porcine or bovine embryo (e.g., by fusing and activating the oocyte), the embryo is transferred to the oviducts of a recipient female, about 20 to 24 hours after activation. See, for example, Cibelli et al., Science, 280:1256-1258, 1998; and U.S. Pat. No. 6,548,741. For pigs, recipient females can be checked for pregnancy approximately 20-21 days after transfer of the embryos.

Standard breeding techniques can be used to create animals that are homozygous for the exogenous nucleic acid or gene modification from the initial heterozygous founder animals. Homozygosity may not be required, however. Transgenic pigs described herein can be bred with other pigs of interest.

In some embodiments, a nucleic acid of interest and a selectable marker can be provided on separate transposons and provided to either embryos or cells in unequal amount, where the amount of transposon containing the selectable marker far exceeds (5-10-fold excess) the transposon containing the nucleic acid of interest. Transgenic cells or animals expressing the nucleic acid of interest can be isolated based on presence and expression of the selectable marker. Because the transposons will integrate into the genome in a precise and unlinked way (independent transposition events), the nucleic acid of interest and the selectable marker are not genetically linked and can easily be separated by genetic segregation through standard breeding. Thus, transgenic animals can be produced that are not constrained to retain selectable markers in subsequent generations, an issue of some concern from a public safety perspective.

Once genome engineered animals have been generated, expression of an exogenous nucleic acid can be assessed using standard techniques. Initial screening can be accomplished by Southern blot analysis to determine whether or not integration of the construct has taken place. For a description of Southern analysis, see sections 9.37-9.52 of Sambrook et al., Molecular Cloning, A Laboratory Manual, second edition, Cold Spring Harbor Press, Plainview; NY., 1989. Polymerase chain reaction (PCR) techniques also can be used in the initial screening. PCR refers to a procedure or technique in which target nucleic acids are amplified. Generally, sequence information from the ends of the region of interest or beyond is employed to design oligonucleotide primers that are identical or similar in sequence to opposite strands of the template to be amplified. PCR can be used to amplify specific sequences from DNA as well as RNA/cDNA, including sequences from total genomic DNA or total cellular RNA. Primers typically are 14 to 40 nucleotides in length but can range from 10 nucleotides to hundreds of nucleotides in length. PCR is described in, for example PCR Primer: A Laboratory Manual, ed. Dieffenbach and Dveksler, Cold Spring Harbor Laboratory Press, 1995. Nucleic acids also can be amplified by ligase chain reaction, strand displacement amplification, self-sustained sequence replication, or nucleic acid sequence-based amplified. See, for example, Lewis, Genetic Engineering News, 12:1, 1992; Guatelli et al., Proc. Natl. Acad. Sci. USA, 87:1874, 1990; and Weiss, Science, 254:1292, 1991. At the blastocyst stage, embryos can be individually processed for analysis by PCR, Southern hybridization and splinkerette PCR (see, e.g., Dupuy et al., Proc Natl Acad Sci USA, 99:4495, 2002).

Expression of a nucleic acid sequence encoding a polypeptide in the tissues of modified animals can be assessed using techniques that include, for example, Northern blot analysis of tissue samples obtained from the animal, in situ hybridization analysis, Western analysis, immunoassays such as enzyme-linked immunosorbent assays, and reverse-transcriptase PCR (RT-PCR).

### Interfering RNAs

A variety of interfering RNA (RNAi) are known. Double-stranded RNA (dsRNA) induces sequence-specific degradation of homologous gene transcripts. RNA-induced silencing complex (RISC) metabolizes dsRNA to small 21-23-nucleotide small interfering RNAs (siRNAs). RISC contains a double stranded RNase (dsRNase, e.g., Dicer) and ssRNase (e.g., Argonaut 2 or Ago2). RISC utilizes antisense strand as a guide to find a cleavable target. Both siRNAs and microRNAs (miRNAs) are known. A method of disrupting a gene in a genetically modified animal comprises inducing RNA interference against a target gene and/or nucleic acid such that expression of the target gene and/or nucleic acid is reduced.

For example, the exogenous nucleic acid sequence can induce RNA interference against a nucleic acid encoding a polypeptide. For example, double-stranded small interfering RNA (siRNA) or small hairpin RNA (shRNA) with complementarity to a target RNA can be used to reduce expression abundance of that RNA. Constructs for siRNA can be produced as described, for example, in Fire et al., Nature, 391:806, 1998; Romano and Masino, Mol. Microbiol., 6:3343, 1992; Cogoni et al., EMBO J., 15:3153, 1996; Cogoni and Masino, Nature, 399:166, 1999; Misquitta and Paterson Proc. Natl. Acad. Sci. USA, 96:1451, 1999; and Kennerdell and Carthew, Cell, 95:1017, 1998. Constructs for shRNA can be produced as described by McIntyre and Fanning (2006) BMC Biotechnology 6:1. In general, shRNAs are transcribed as a single-stranded RNA molecule containing complementary regions, which can anneal and form short hairpins.

The probability of finding a single, individual functional siRNA or miRNA directed to a specific gene is high. The predictability of a specific sequence of siRNA, for instance, is about 50% but a number of interfering RNAs may be made with good confidence that at least one of them will be effective.

Embodiments include an in vitro cell, an in vivo cell, and a genetically modified animal such as a livestock animal that express an RNAi directed against a gene, e.g., a gene selective for a developmental stage. The RNAi may be, for instance, selected from the group consisting of siRNA, shRNA, dsRNA, RISC and miRNA.

### Inducible Systems

An inducible system may be used to control expression of a gene. Various inducible systems are known that allow spatiotemporal control of expression of a gene. Several have been proven to be functional in vivo in transgenic animals. The term inducible system includes traditional promoters and inducible gene expression elements.

An example of an inducible system is the tetracycline (tet)-on promoter system, which can be used to regulate transcription of the nucleic acid. In this system, a mutated Tet repressor (TetR) is fused to the activation domain of herpes simplex virus VP16 trans-activator protein to create a tetracycline-controlled transcriptional activator (tTA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP). Expression is induced by treatment with ecdysone or an analog of ecdysone such as muristerone A. The agent that is administered to the animal to trigger the inducible system is referred to as an induction agent.

The tetracycline-inducible system and the Cre/loxP recombinase system (either constitutive or inducible) are among the more commonly used inducible systems. The tetracycline-inducible system involves a tetracycline-controlled transactivator (tTA)/reverse tTA (rtTA). A method to use these systems in vivo involves generating two lines of genetically modified animals. One animal line expresses the activator (tTA, rtTA, or Cre recombinase) under the control of a selected promoter. Another set of transgenic animals express the acceptor, in which the expression of the gene of interest (or the gene to be modified) is under the control of the target sequence for the tTA/rtTA transactivators (or is flanked by loxP sequences). Mating the two strains of transgenic animals provides control of gene expression.

The tetracycline-dependent regulatory systems (tet systems) rely on two components, i.e., a tetracycline-controlled transactivator (tTA or rtTA) and a tTA/rtTA-dependent promoter that controls expression of a downstream cDNA, in a tetracycline-dependent manner. In the absence of tetracycline or its derivatives (such as doxycycline), tTA binds to tetO sequences, allowing transcriptional activation of the tTA-dependent promoter. However, in the presence of doxycycline, tTA cannot interact with its target and transcription does not occur. The tet system that uses tTA is termed tet-OFF, because tetracycline or doxycycline allows transcriptional down-regulation. Administration of tetracycline or its derivatives allows temporal control of transgene expression in vivo. rtTA is a variant of tTA that is not functional in the absence of doxycycline but requires the presence of the ligand for transactivation. This tet system is therefore termed tet-ON. The tet systems have been used in vivo for the inducible expression of several transgenes, encoding, e.g., reporter genes, oncogenes, or proteins involved in a signaling cascade.

The Cre/lox system uses the Cre recombinase, which catalyzes site-specific recombination by crossover between two distant Cre recognition sequences, i.e., loxP sites. A DNA sequence introduced between the two loxP sequences (termed foxed DNA) is excised by Cre-mediated recombination. Control of Cre expression in a transgenic animal, using either spatial control (with a tissue- or cell-specific promoter) or temporal control (with an inducible system), results in control of DNA excision between the two loxP sites. One application is for conditional gene inactivation (conditional knockout). Another approach is for protein over-expression, wherein a floxed stop codon is inserted between the promoter sequence and the DNA of interest. Genetically modified animals do not express the transgene until Cre is expressed, leading to excision of the floxed stop codon. This system has been applied to tissue-specific oncogenesis and controlled antigen receptor expression in B lymphocytes. Inducible Cre recombinases have also been developed. The inducible Cre recombinase is activated only by administration of an exogenous ligand. The inducible Cre recombinases are fusion proteins containing the original Cre recombinase and a specific ligand-binding domain. The functional activity of the Cre recombinase is dependent on an external ligand that is able to bind to this specific domain in the fusion protein.

Embodiments include an in vitro cell, an in vivo cell, and a genetically modified animal such as a livestock animal that comprise a gene under control of an inducible system. The genetic modification of an animal may be genomic or mosaic. The inducible system may be, for instance, selected from the group consisting of Tet-On, Tet-Off, Cre-lox, and Hif1alpha. An embodiment is a gene set forth herein.

### Dominant Negatives

Genes may thus be disrupted not only by removal or RNAi suppression but also by creation/expression of a dominant negative variant of a protein which has inhibitory effects on the normal function of that gene product. The expression of a dominant negative (DN) gene can result in an altered phenotype, exerted by a) a titration effect; the DN PASSIVELY competes with an endogenous gene product for either a cooperative factor or the normal target of the endogenous gene without elaborating the same activity, b) a poison pill (or monkey wrench) effect wherein the dominant negative gene product ACTIVELY interferes with a process required for normal gene function, c) a feedback effect, wherein the DN ACTIVELY stimulates a negative regulator of the gene function.

### Founder Animals, Animal Lines, Traits, and Reproduction

Founder animals (FO generation) may be produced by cloning and other methods described herein. The founders can be homozygous for a genetic modification, as in the case where a zygote or a primary cell undergoes a homozygous modification. Similarly, founders can also be made that are heterozygous. The founders may be genomically modified, meaning that the cells in their genome have undergone modification. Founders can be mosaic for a modification, as may happen when genes are edited or modified in one of a plurality of cells in an embryo, typically at a blastocyst stage. Progeny of mosaic animals may be tested to identify progeny that are genomically modified. An animal line is established when a pool of animals has been created that can be reproduced sexually or by assisted reproductive techniques, with heterozygous or homozygous progeny consistently expressing the modification.

In livestock, many alleles are known to be linked to various traits such as production traits, type traits, workability traits, and other functional traits. Artisans are accustomed to monitoring and quantifying these traits, e.g., Visscher et al., Livestock Production Science, 40:123-137, 1994; U.S. Pat. No. 7,709,206; U.S. 2001/0016315; U.S. 2011/0023140; and U.S. 2005/0153317. An animal line may include a trait chosen from a trait in the group consisting of a production trait, a type trait, a workability trait, a fertility trait, a mothering trait, and a disease resistance trait. Further traits include expression of a recombinant gene product.

### Recombinases

Embodiments of disclosure include administration of a targeted nuclease system with a recombinase (e.g., a RecA protein, a Rad51) or other DNA-binding protein associated with DNA recombination. A recombinase forms a filament with a nucleic acid fragment and, in effect, searches cellular DNA to find a DNA sequence substantially homologous to the sequence. For instance, a recombinase may be combined with a nucleic acid sequence that serves as a template for HDR. The recombinase is then combined with the HDR template to form a filament and placed into the cell. The recombinase and/or HDR template that combines with the recombinase may be placed in the cell or embryo as a protein, an mRNA, or with a vector that encodes the recombinase. The disclosure of U.S. 2011/0059160 (U.S. patent application Ser. No. 12/869,232) is hereby incorporated herein by reference for all purposes; in case of conflict, the specification is controlling. The term recombinase refers to a genetic recombination enzyme that enzymatically catalyzes, in a cell, the joining of relatively short pieces of DNA between two relatively longer DNA strands. Recombinases include Cre recombinase, Hin recombinase, RecA, RAD51, Cre, and FLP. Cre recombinase is a Type I topoisomerase from P1 bacteriophage that catalyzes site-specific recombination of DNA between loxP sites. Hin recombinase is a 21 kD protein composed of 198 amino acids that is found in the bacteria Salmonella. Hin belongs to the serine recombinase family of DNA invertases in which it relies on the active site serine to initiate DNA cleavage and recombination. RAD51 is a human gene. The protein encoded by this gene is a member of the RAD51 protein family which assists in repair of DNA double strand breaks. RAD51 family members are homologous to the bacterial RecA and yeast Rad51. Cre recombinase is an enzyme that is used in experiments to delete specific sequences that are flanked by loxP sites. FLP refers to Flippase recombination enzyme (FLP or Flp) derived from the 2μ plasmid of the baker's yeast Saccharomyces cerevisiae.

Herein, “RecA” or “RecA protein” refers to a family of RecA-like recombination proteins having essentially all or most of the same functions, particularly: (i) the ability to position properly oligonucleotides or polynucleotides on their homologous targets for subsequent extension by DNA polymerases; (ii) the ability topologically to prepare duplex nucleic acid for DNA synthesis; and, (iii) the ability of RecA/oligonucleotide or RecA/polynucleotide complexes efficiently to find and bind to complementary sequences. The best characterized RecA protein is from E. coli; in addition to the original allelic form of the protein a number of mutant RecA-like proteins have been identified, for example, RecA803. Further, many organisms have RecA-like strand-transfer proteins including, for example, yeast, Drosophila, mammals including humans, and plants. These proteins include, for example, Rec1, Rec2, Rad51, Rad51B, Rad51C, Rad51D, Rad51E, XRCC2 and DMC1. An embodiment of the recombination protein is the RecA protein of E. coli. Alternatively, the RecA protein can be the mutant RecA-803 protein of E. coli, a RecA protein from another bacterial source or a homologous recombination protein from another organism.

### Compositions and Kits

The present disclosure also provides compositions and kits containing, for example, nucleic acid molecules encoding site-specific endonucleases, CRISPR, Cas9, ZNFs, TALENs, RecA-gal4 fusions, polypeptides of the same, compositions containing such nucleic acid molecules or polypeptides, or engineered cell lines. An HDR may also be provided that is effective for introgression of an indicated allele. Such items can be used, for example, as research tools, or therapeutically.

The present disclosure pairs GST and BC techniques with germline-ablated, DAZL null pigs to create a DAZL breeding platform for the production of high mortality or failure to thrive gene edited animals for models of disease and organ production. GST and BC have enabled genotype/phenotype rescue and permitted germline transmission in the past, they were encumbered by low or highly variable rates of transmission of the desired genotype, considerably diminishing reliability/consistency of use.

Using germline-ablated DAZL null boars for GST or DAZL null embryos for chimera production, the germline is formed with only the desired genotype. The significance of this innovation becomes evident when considering the opportunities enabled and the cost savings of deploying the DAZL platform. In simple heterozygous disease models like dilated cardio myopathy, GST cuts the cost of producing one animal in half whereas BC with male and female SCID lines can boost production 16-fold versus heterozygous intercross. Certainly, as the number of modified loci increases, the fold benefit of using the DAZL platform increases exponentially. Hence; this platform enables the production of a wide variety of new, usable and very powerful animal disease models, and will transform the approach and scalability of exogenic production of human organs and tissues.

### GST for Line Rescue and Increased Efficiency of Model Propagation

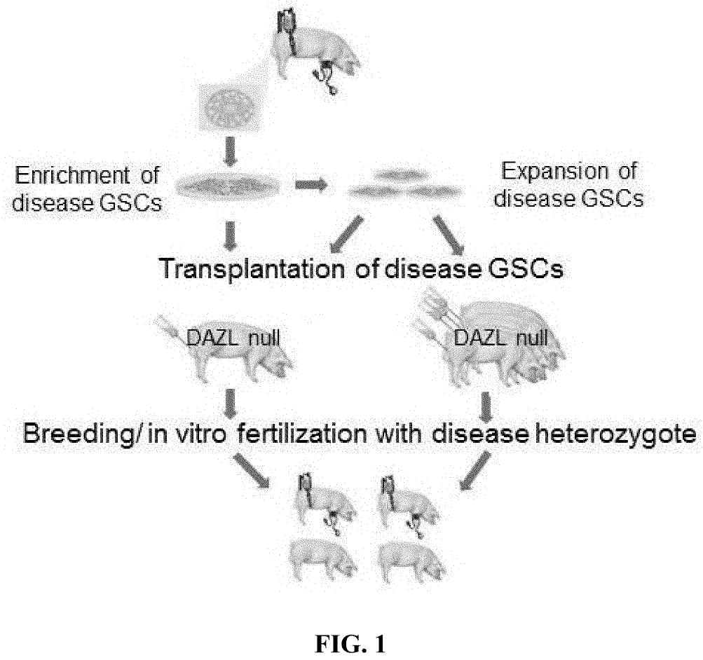

Spermatogenesis is the highly coordinated process of spermatogonial (germline) stem cell renewal and differentiation to produce spermatozoa. Brinster and colleagues first demonstrated that transplantation of germline stem cells (GSCs) from fertile donor mice to the testes of infertile recipient mice resulted in donor-derived spermatogenesis and germline transmission. The GST technique has been adapted for large animals including goats, pigs, sheep and cattle. GST enables the germline rescue of valuable disease or lineage/organogenesis-deficient swine models affected by prepubertal mortality or an inability to sexually reproduce as adults (FIG. 1).

Properties of GST recipients are integral to success. Characteristics of the recipient animal, including capacity for endogenous spermatogenesis and age, influence the efficiency and reproducibility of GSC transplantation and colonization. Unlike in rodents, immune rejection of GSCs has not been observed in large animals including pigs and cattle. Thus, a single breed of swine could be used as a universal recipient of GSCs. However, when GSCs are transplanted into wildtype recipients, donor cells must compete with endogenous GSCs for stem cell niches which can result in poor colonization of the germline stem cells. Studies show that donor GSC colonization and spermatogenesis following GST are significantly improved by expanding the availability of the recipient stem cell niche. Chemical or radiation-induced ablation of recipient endogenous GSCs using the chemotherapy agent busulfan or irradiation treatments have been effective. However, these treatments are temporary and effectiveness often varies considerably from one animal to another, resulting in unpredictable recovery of endogenous spermatogenesis after transplantation. Furthermore, the application of radiation to large animals requires specialized equipment not readily accessible for use in large animals while busulfan treatment is associated with systemic cytotoxic effects and adverse alteration of the recipient microenvironment. Using these ablation strategies, greater than 50% of donor-derived offspring can be achieved in rodents while the highest reported in large animals using irradiation is 15% (sheep) and is typically below 30% in pigs with busulfan treatment.

Genetic ablation of endogenous spermatogenesis represents a failsafe method for elimination of recipient GSCs and has been extremely successful in c-kit−/− mice and Dazl −/− mice and rats. GST of normal GSCs into Dazl-deficient rats restored fertility and resulted in 100% of germline transmission of donor alleles to offspring by natural mating. Unlike in laboratory rodents, large animal models with genetically impaired spermatogenesis are very limited. Recently, Park and colleagues generated germline ablated male pigs by disruption of the NANOS2 gene. Although the NANOS2 null males lack germ cells when evaluated in adulthood, their suitability for GST has not been publicized and it is unknown whether the germ cell niche is vacant at transplant age (10-12 weeks). Also, since NANOS2 is dispensable for female fertility, it would not be suitable for rescue of the female germline by blastocyst complementation. Thorough characterization of the DAZL null phenotype in boars revealed a complete lack of GSCs by 11 weeks of age while the seminiferous tubule morphology remained intact suggesting that these DAZL null boars are ideal hosts for GST.

Enrichment and Expansion of Germ Cells. In addition to preparation of the recipient, the relative number of donor cells and enrichment of GSCs also have a significant effect on GST success and spermatogenesis efficiency. To increase colonization efficiency, novel GSC enrichment techniques disclosed herein can be used. Utilizing the differential adhesion properties of porcine germ cells and somatic cells to plastic, >10-fold enrichment of GSCs has been achieved by the applicants. Similar enrichment of porcine GSCs by incubating the initial cell suspension in stirred bioreactor culture have been obtained. With this technique somatic cells form clumps that are easily removed while germ cells remain in suspension. In mice, GST has benefited from the progress made through in vitro expansion of GSCs. Mice GSCs can be expanded in culture reducing the number of donor testes initially needed and increasing efficiency of colony expansion by GST. Maintenance and proliferation of non-rodent germ cells in culture has so far met with limited success, partially due to the lack of highly enriched starting populations. Disclosed herein are culture conditions that support proliferation of porcine germ cells in culture.

### Blastocyst Complementation for Phenotypic and Germline Rescue of Lineage/Organogenesis-Deficient Lines

Inactivation of genes critical for lineage specification and organogenesis during development often results in the failure of specific cell lineage(s) or organs to develop, creating a vacant developmental niche. These vacant “niches” can be “complemented” with wildtype donor pluripotent stem cells (at the blastocyst stage) resulting in donor-derived cell lineages or organs within a fertile host. The BC approach has produced functional lymphocytes, pancreas, kidney and liver in rodents. In cattle, BC has been used to generate exogenous germ cells in the ovaries of a gametogenesis-deficient females. Recently, in an initial step towards the in vivo production of xenogeneic functional organs in a large mammalian system, allogenic BC was used to generate functional pancreata in pigs that grew into fertile adults. Disclosed herein, BC restores deficient cell types in a number of genotypes including lymphocytes, vasculature, dopamine neurons, liver and skeletal muscle in singly or multi-edited pigs (data not shown). Exogenic production of human organs is one key objective, but with the frequent requirement for multiple gene edits, SCNT is the only feasible way to generate these lines, significantly impeding development of this exciting solution for overcoming the shortage of transplantable organs.

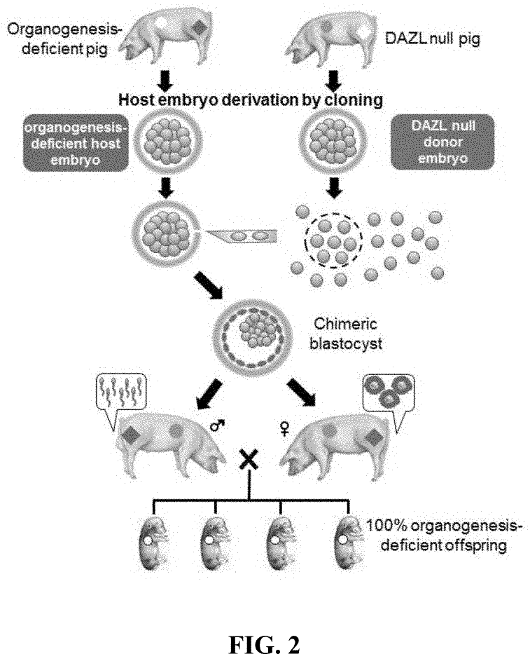

Despite inefficiencies, SCNT remains the most common method for generating lineage/organogenesis-disabled pigs. With the recent advances in application and efficiency of genome-editing techniques, TALEN and CRISPR zygote injections have been used as alternatives to SCNT for creation of lineage/organogenesis-deficient blastocysts. However, due to random indel formation during DNA repair, these approaches can result in in-frame mutations that fail to disrupt gene function/organogenesis as well as result in allelic mosaicism making the precise genotype unknown. Hence; the system is unpredictable and not scalable. Alternatively, lineage/organogenesis-deficient heterozygous founders established by SCNT could breed to produce homozygous embryos; however, a maximum of 25% of the embryos would be useful for BC, a fraction that sharply declines when segregating more than one locus. Better propagation methods are required to make exogenic organ production a reality. Progress towards the exogenic production of human organs in pigs for transplantation will require an ability to more efficiently generate well-characterized, lineage/organogenesis-deficient embryos for BC. Germline cell-deficient DAZL male swine are ideal donors for BC of lineage/organogenesis-deficient hosts. As donors the DAZL null cells can rescue the lethal phenotype, but because they do not contribute to the adult germline, only gametes carrying the lineage/organogenesis-deficient genotype are produced. Furthermore, data suggest that DAZL null females also lack germ cells enabling complementation of germline in both sexes, increasing the number of useful blastocysts for complementation to 100% (FIG. 2).

The creation and propagation of biomedical animals and, in particular, swine is hindered by an inability to overcome substantial inefficiencies related to animal development, reproduction and lethal phenotypes. The DAZL platform disclosed herein will permit for the first time efficient propagation of congenital disease, lineage/organogenesis-deficient and multi-genic alleles and establish the basis for a production method that does not rely on inconsistencies produced by cloning (SCNT). The DAZL null, germline ablated pig, combined with GST and BC is a key innovation.

While GST has been shown in germ cell intact swine, the transmission of donor genetics was deficient due to the competitive advantage of endogenous germs cells versus the transplanted donor cells. In contrast, using GST in germline ablated DAZL null rats and mice, hosts become fertile and have 100% donor derived spermatogenesis. It is believed the same will hold true for GST in DAZL null boars, and will provide a significant advantage when breeding low viability or multi-edited lines. This breeding advantage is one solution to one of the most vexing problems associated with propagating disease model lines; inefficiency in production leading to large, expensive breeding herds for minimal salable output. As in the DCM example, only heterozygotes reach breeding age due to disease severity, but many homozygotes do live long enough to enable germ cell harvest and transplantation into a DAZL null boar. Breeding from this transplanted boar with heterozygous females results in 50% salable offspring (homozygotes) versus the 25% from traditional heterozygous intercross. This simple change provides the ability to cut the breeding herd in half representing a 50% reduction in cost of goods. In the case of multigene recessive disorders with 2 or 3 genes, the GST reduction in cost of goods is 75 and 87.5% respectively. Hence; the DAZL platform is a key innovation that enables production of complex disease model lines that before would have been cost prohibitive to produce. Another innovative aspect of the DAZL platform and GST, is the ability to generate novel animal models through transplantation of in vitro gene-modified germline stem cells to DAZL null males.

Whereas the GST using the DAZL platform in itself is highly innovative for enhanced breeding of swine models, perhaps a greater potential comes from pairing the innovation of DAZL null, germline-deficient animals with blastocyst complementation. The basis of this platform is chimera production, which has been routine in mice to generate germline competent, gene targeted lines. On repeated occasions, germline competency of pig chimera has also been demonstrated in males and females. Blastocyst complementation is simply a chimera where either the donor cells, host embryo, or both are engineered to lack a certain cell type, tissue or organ (lineage/organogenesis-deficient). When put together in a chimera, the deficiency of either the host or the donor cells is complemented by the other, filling the void left vacant in either line and rescuing these often-lethal phenotypes. One innovation inherent to the DAZL platform for BC is the ability of DAZL null cells in the chimera to rescue the phenotypes of lineage/organogenesis-deficient lines without contributing to the germline. This solves the problem of mosaic germline between traditional chimeras made between wild-type animals and those with lineage/organogenesis-deficiencies. The DAZL blastocyst complementation platform has the potential to enable production of high quality, in vivo produced knockout blastocysts where ALL have the desired genotype, even if multiple genes are inactivated. These far superior, in vivo produced lineage/organogenesis-deficient embryos will form the cornerstone in production of human cells and organs in pigs for human therapeutics. Blastocyst complementation with the DAZL platform can be used to address a current need, phenotypic and germline rescue of the inventors B-, T-, and NK-cell deficient severe combined immunodeficiency (SCID) line of pigs.

The flexibility of deploying the DAZL platform using GST and/or BC is advantageous due to the strengths and limitations of each approach. A strength of GST is it is technically simpler than BC. In addition, many disease models created (i.e. DCM, polycystic kidney diseases and cancers) may not be readily rescued with BC due to phenotypic cell-autonomous effects. Other current disease models in animals and especially pigs, could benefit from enhanced reproduction using the GST platform, including cystic fibrosis, colon cancer, familial hypercholesterolemia and FAH deficiency. On the other hand, BC benefits from the immense power of rescuing male and female lethal genotypes, while permitting 100% of the germline to transmit the desired genotype. Establishing a breeding herd using BC is costlier upfront than GST; hence, propagation with BC will have the greatest impact when propagating lines edited at multiple loci and/or lineage/organogenesis-deficient lines. Both aspects of the DAZL breeding platform will efficiently produce animal models of human diseases and provide a distinct advantage towards the exogenic production of human cells/organs for regenerative medicine.

Various exemplary embodiments of devices and compounds as generally described above and methods according to this disclosure, will be understood more readily by reference to the following examples, which are provided by way of illustration and are not intended to be limiting of the disclosure in any fashion.

### EXAMPLES

### Example 1. Characterization of Male Reproductive Phenotype of DAZL^{-/-} Pigs

The founder DAZL^{-/-} boars were developed using TALEN stimulated homology dependent repair followed by SCNT⁴⁸. Aside from some minor flexor tendon abnormalities common to cloning⁴⁹, there was no visible phenotype in the founders and they displayed typical boar behavior; aggressiveness, strong odor, and mounting at the onset of puberty. Once they reached 7 months of age, the boars were trained for semen collection. In a blind evaluation, microscopic analysis of 3-serial ejaculates collected from the DAZL^{-/-} boars showed no detectable sperm. These findings were confirmed in ejaculates concentrated by centrifugation (data not shown).

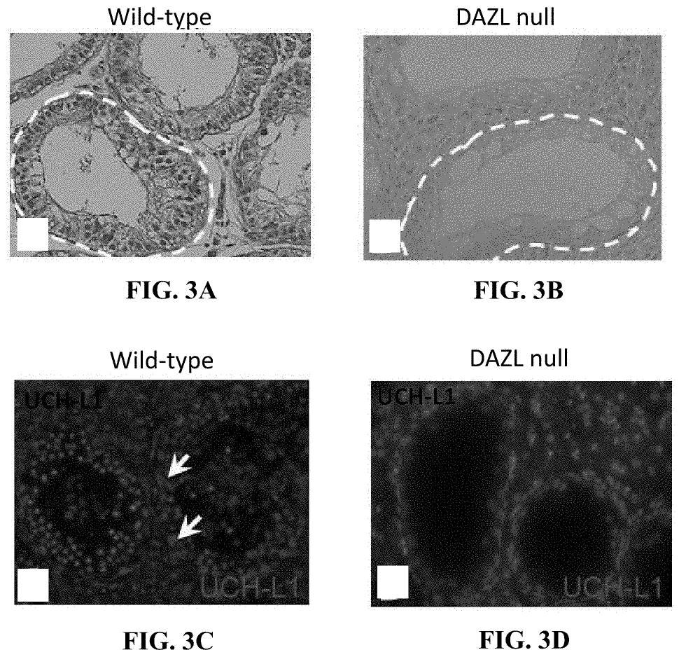

Histological evaluation of cross sections of adult DAZL^{-/-} testes revealed intact seminiferous tubules completely devoid of germ cells within the lumen suggesting spermatogenic failure (FIG. 3). To further characterize the DAZL^{-/-} spermatogenic failure phenotype, cross sections from adult and 10 week and DAZL^{-/-} testes were analyzed for expression of germ cell and somatic cell markers by immunohistochemistry (FIGS. 3 & 4). Consistent with the absence of germ cells in seminiferous tubules in hematoxylin and eosin stained sections, no expression of type A spermatogonia cell marker UCH-L1³¹ was observed in adult (FIG. 3) or 10-week-old testes sections. Taken together, this indicates that the failure of spermatogenesis in the DAZL^{-/-} boars is due to the absence of germline stem cells. In Dazl knockout mice, the loss of spermatogenesis coincides with a 3.4-fold reduction in testis mass compared to wildtype⁵⁰. Surprisingly, in DAZL^{-/-} porcine testis a reduction in mass was not observed.

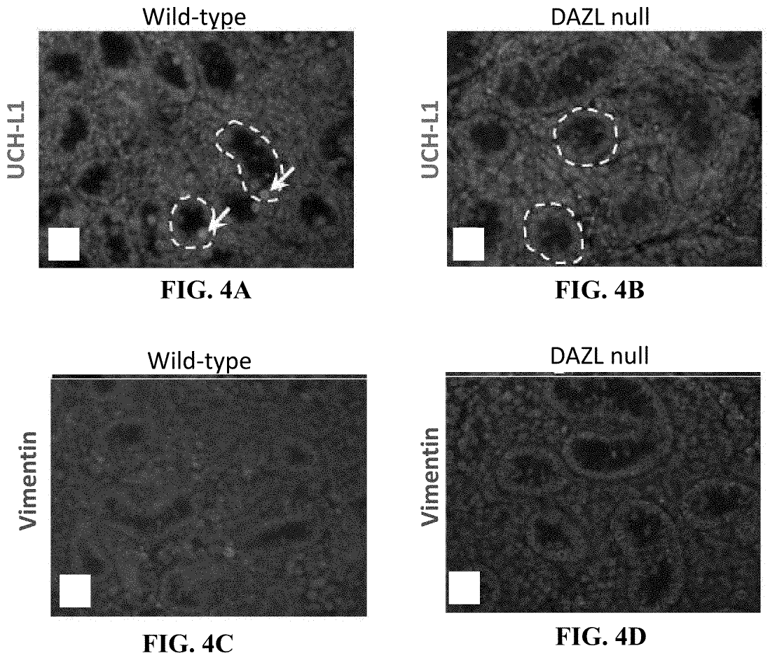

Within the seminiferous tubules, somatic Sertoli cells provide structural and functional support to germ cells and are required for spermatogenesis⁵¹. To examine the effect of DAZL^{-/-} on Sertoli cell morphology 10 wk-old DAZL^{-/-} and WT testes sections were labeled for vimentin, an intermediate filament marker and indicator of the structural integrity of the seminiferous epithelium⁵² (FIG. 4). The loss of vimentin expression is associated with spermatogenic dysfunction. Vimentin expression in DAZL^{-/-} testes was similar to that observed in WT testes confirming that although germ cells are absent in the DAZL^{-/-} testes, the seminiferous tubule morphology remains intact. The absence of germ cells by 10 weeks of age in the DAZL^{-/-} testes and the preservation of tubule morphology suggest that the DAZL^{-/-} testes is an ideal environment for GST or blastocyst complementation.

### Example 2. Production of Heterozygous KO DAZL^{+/-} Male and Female Swine By TALEN-Stimulated HR and SCNT

Male and female cellular pools consisting of sequence validated DAZL^{+/-} clones with confirmed mutation were used to generate a breeding herd of DAZL^{+/-} swine by cloning. To ensure the establishment of a heterozygous breeding herd, DAZL^{+/-} males were left intact instead of castrating at 10 wks. The DAZL^{+/-} intact males have been useful for breeding and transmit the knockout allele at a predicted rate. When bred to DAZL^{+/-} females, DAZL null animals were produced. Similar to the ovary phenotype in DAZL null mice, the DAZL null females presented with one or both micro-ovaries that lacked follicles and ova.

Ejaculates were collected from two, 8-month DAZL^{+/-} boars and evaluated. Three separate ejaculate samples from each boar were cryopreserved and analyzed for post thaw characteristics. Ejaculates from the first boar showed poor post thaw characteristics and were not used for artificial insemination. Ejaculates from the second of the DAZL^{+/-} boars showed good pre- and post-thaw characteristics and were used for artificial insemination resulting in successful pregnancies and piglets. Hence; DAZL^{+/-} animals are fertile, enabling scaled up production by standard breeding to serve as host animals for the GST platform.

### Example 3. Optimizing GST In DAZL Null Boars Using Genetically Similar And Divergent GSCs

Only recently has GST been demonstrated in large animals, including swine. Due to the lack of genetically-derived germ cell-deficient hosts, these experiments were performed in busulfan-treated, irradiated or untreated recipients^{14,15}. This approach has been used to generate transgenic swine embryos by viral transduction of germline stem cells followed by transplantation into busulfan-treated and untreated recipients⁵³. However, germ cell transplantation of TALEN edited GSCs into untreated recipient testes failed to produce detectable levels of genome-edited and unmodified donor-derived sperm using SNP sequencing—presumably due to an inability to compete with native GSCs after the stressful transfection process (data not shown). DAZL null boars are a favorable alternative to the currently used approach for recipient preparation. Optimization of GST in the context of the germ cell-deficient DAZL null boars is ongoing. In previous GST experiments in swine, dosage of GSCs transplanted was extrapolated from work in mice and transmission of the donor genotype was achieved following transplantation of 30 million donor cells to untreated or busulfan-treated testes⁵³. This high number may not be required in DAZL null boars since no native GSCs are present to compete. In mice, germ cell transplantation had about 10% colonization efficiency⁵⁴ and restoration of fertility required spermatogenesis in about 20% of the testis⁵⁵. Similar information is not available for large animals. Therefore, a range of GSC transplantation dosages are evaluated. Transplantations are performed in prepubertal, ˜10-week-old DAZL null boars because the lumen of the seminiferous tubules is formed by this age. Also, while previous GST experiments in large animals have not reported GSC rejection when transplanted into an immunocompetent recipient, the stochastic success rate justifies controlled evaluation of GST using divergent donor breeds, such as Ossabaw.

Transplantation of strain-matched and Ossabaw germ cells to DAZL null boar recipients. DAZL^{+/-} males and females are bred by artificial insemination to generate DAZL^{-/-} recipient boars. Donor cells are isolated from 10 wk old DAZL^{+/+} testes obtained from litter mates of the recipient boars using the standard protocol⁵³. 30 million, 3 million, 300,000 or 30,000 cells (from a single round of-GSC preparations) are transplanted to each testis of individual recipients. This process is repeated to generate 5 recipients for each number of cells transplanted to account for variability in donor cell preparations and recipient testes colonization. For the transplantation of Ossabaw germ cells, donor cells are isolated from 10 wk old wildtype Ossabaw testes. 30 million cells and the lowest dosage of GSCs shown to result in sperm in the ejaculates from experiment 1 after transplantation of WT GSCs are transplanted. Transplants are performed to each testis of 5 individual recipients per dosage. The GST procedure is performed by ultrasound guided injection in rete testes of 2 mo. old DAZL^{-/-} recipients as previously describer. Approximately 3 ml of cell suspension is infused into each testis with a flow rate of 0.5-1 ml/min. After cell transplantation, testes are returned to the scrotum, the scrotal skin is closed, and animals are allowed to recover.

### Analysis of DAZL Null GST Recipients

Recipient pigs are maintained through sexual maturity and trained for semen collection. Semen is collected beginning 3 mo. post transplantation and continued weekly until 1 year of age. Ejaculates from each recipient are analyzed for sperm concentration, morphology and viability as indicators of artificial insemination competency. Microsatellite markers from ejaculates are analyzed to determine if all sperm are donor-derived. Briefly, genomic DNA isolated from individual ejaculates, 3 or more per animal, is used for PCR amplification of identified microsatellite markers and quantified by Illumina amplicon sequencing. At ˜1-year recipients are sacrificed for quantification of donor cell colonization and characterization of spermatogenesis using histology and immunohistochemistry. Testes tissue is collected adjacent, medial and distal to rete injection site. Morphological analysis of H & E stained sections include quantification of meiotic and non-meiotic germ cells and percentage of tubules with germ cells. The expression pattern of germ cell and somatic cell specific proteins in adult porcine testes have been demonstrated. Spermatogenic progression is characterized by the following markers using indirect immunofluorescence: Undifferentiated type A spermatogonia—UCH-L1. Differentiating type A spermatogonia—Dazl, c-kit⁵⁶. Spermatocytes SCP3, gamma H2AX⁵⁷. Testicular somatic cells are identified by expression of Gata4 (Sertoli cells) and STARr/P450scc (Leydig cells)⁵⁸. These experiments identify a feasible dosage of GSC that results in sufficient sperm production for downstream application.

In Vitro Fertilization with GST-Derived Sperm and Embryo Analysis for Donor-Derived Genotype

Abattoir oocytes are in vitro matured and fertilized with semen collected from GST recipients as previously established⁵³. Briefly, matured oocytes are denuded of surrounding cumulus cells, washed and transferred to IVF dishes. Sperm are prepared by density separation using a Percoll gradient followed by pelleting and washing. Sperm are added to oocytes for a final sperm concentration of 250 sperm/ul. At day 6 of development genomic DNA is isolated from embryos and analyzed for the donor-derived genotype as conducted previously⁴⁶. GST ejaculates demonstrating successful IVF and suitable semen quality and characteristics are used for artificial insemination. Fertile sows are inseminated with 2 billion live sperm in 100 ml per insemination and pregnancy checks are conducted at days 25, 50, and 100.

Previous experiments with GST in Dazl-deficient rodents, indicates that colonization and spermatogenesis from DAZL null recipient boars transplanted with 30 million DAZL^{+/+} or wildtype Ossabaw cells is expected. As donor material for GSC isolation may be limiting for certain disease models, the experiment is designed to establish the dosage required to effectively restore spermatogenesis. The series of 10, 100 and 1000-fold reduction in the number of transplanted cells from the applicant's typical protocol is expected to result in differential rates of colonization and spermatogenesis. Therefore, assuming there is a minimum number of cells, animals transplanted with reduced numbers of cells may not have sufficient spermatogenesis for standard breeding. However, for extremely valuable lines, IVF, deep intrauterine insemination or surgical fallopian tube insemination is an option due to the lower sperm counts required^{59,60}. These experiments also reveal if there is a strain difference in colonization efficiency and hence inform the required dosage for transplantation from Ossabaw donors.

### Example 4. Expanding the Feasible Donor Age for GSC Isolation

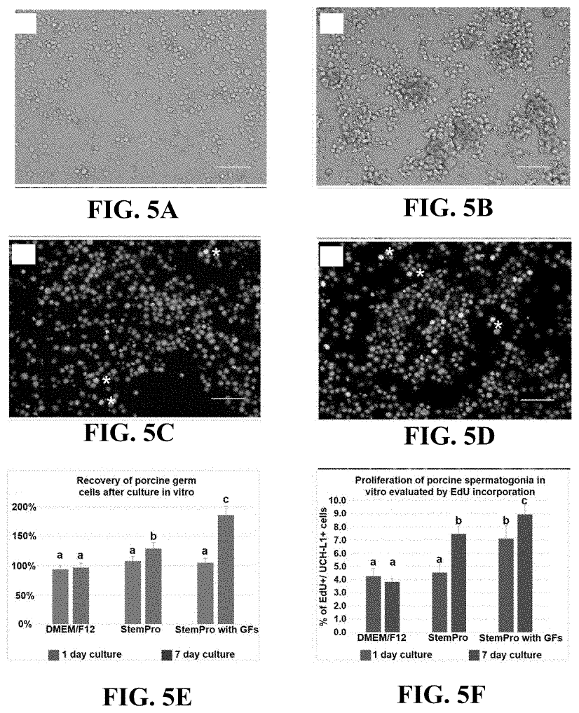

Donor age affects the number of germ cells present in the testis and the relative number of putative GSCs in the total cell population²¹. Attempts to isolate porcine germ cells usually use neonatal donor testes⁶¹. It was previously established, that harvesting donor germ cells from animals just before puberty maximizes the relative number of germ cells collected³¹. In preliminary work, donor age did not affect efficiency of germ cell enrichment by differential adhesion to plastic in sequential subculture of non-adherent cells from neonatal (1 wk old), 3-week or prepubertal (8 wk old) testes donors making this a promising method to obtain large scale enrichment of porcine germ cells from donors of varying ages. Recently, similar enrichment of spermatogonia from 1 wk old testes when incubating the initial cell suspension in stirred bioreactor culture was observed³². However, in these previous experiments spermatogonia were harvested from multiple donor testes, which is less feasible for some swine models of disease. Limited availability of harvestable GSCs may be mitigated by expansion of GSCs in vitro prior to transplantation. Although robust GSC expansion has been demonstrated in mice, efficient expansion of porcine germ cells in culture has been limited. Preliminary Culture conditions have been identified that for the first-time support proliferation of germ cells from 8 wk old pigs in culture with StemPro medium supplemented with GDNF, GFRa1 and EGF growth factors (FIG. 5). The applicants will extend the applicability of the GST platform by optimizing germ cell enrichment and expansion techniques using testes from different age wildtype donors. Specifically, the number of spermatogonia per gram of testis from 1 wk, wk and 8 wk donors is evaluated following enrichment using differential plating and/or stirred bioreactor culture. owing enrichment, porcine GSCs from each donor age is cultured and evaluated for proliferation over time.

Enrichment of Porcine GSCs from 1 Wk, 4 wk and 8 wk Donors

Donor cells are harvested from testes obtained at castration of wildtype 1 wk, 4 wk or 8 wk old pigs. Single-cell suspensions are prepared by sequential enzymatic digestion as described¹⁵. Differential plating for enrichment of pig germ cells is performed as described with some modifications³⁴. After 3 rounds of differential plating, cells are plated again onto 100 mm plates in DMEM/F12 with 5% FBS for 8 min at RT and cell suspensions are gently collected from the top to remove remaining cell debris, red blood cells and other small somatic cells. This procedure results in cell suspensions containing >70% UCH-L1+ spermatogonia. UCH-L1 is specifically expressed in undifferentiated type A spermatogonia³¹. Cell suspensions (5×10⁶ cells/ml) are cultured in DMEM and 5% FBS in stirred bioreactors and agitated at 100 rpm for 48 hours. Every 24 hours, cell suspensions are filtered through a 40 μm mesh to remove large aggregates of somatic cells, followed by one round of differential plating as described³².

### Identification and Characterization of Enriched GSCs

Immunofluorescence (IF) is used to identify and quantify cells on the cellular level and distinguish germ cells from somatic cells using the markers described in Aim 1 analysis. Success is defined as isolation of cell populations containing >70% UCH-L1+ germ cells. Quantitative RT-PCR is used to verify results on the cell population level. Germ cell viability and yield per gram testis tissue are analyzed for each donor age (n=5 replicates/age) and approach (differential plating and/or stirred suspension culture).

### Expansion of Porcine GSCs In Vitro

Cells from 1 wk, 4 wk and 8 wk old boar testes (5 replicates per donor age) Are enriched for GSCs as described above. GSCs from each enrichment condition and age are cultured at 37° C. in 5% CO₂ in air in 6-well plates for up to 28 days in StemPro medium (Invitrogen) supplemented with 0.5% FBS, 0.1% BSA, 2 mM L-glutamine, MEM Non-Essential Amino Acids (Invitrogen), 10 μm 2-mercaptoethanol (Invitrogen), 10 μg/ml Insulin (Sigma), 40 ng/ml GDNF, 25 ng/ml GFRa1, and 20 ng/ml EGF (all growth factors from R & D systems) that in preliminary experiments provided the highest proliferation of GSCs. The culture of GSCs in 10% oxygen, on mitotically inactivated pig fetal fibroblasts (PFF) as feeders, and with addition of various growth factors and signaling molecules including FGF2, CSF-1 and Wnt to the culture medium^{62,63} is investigated. Cells cultured in StemPro medium serve as baseline control.

### Analysis of Porcine GSC Expansion

After 7, 14, 21 and 28 days in culture, a sample of cells is collected, plated on poly-2-lysine-coated chamber slides and evaluated for the presence and number of undifferentiated germ cells by IF for UCH-L1. Differentiating germ cells are identified by expression of c-kit (Santa Cruz) and Sertoli cells based on expression of GATA-4³¹. Proliferating germ cells are identified by incorporation of EdU (Invitrogen), expression of PCNA (DAKO) or Ki67 (eBioscience). Maintenance and proliferation of germ cells over time is compared within and between donor age and between culture conditions. Success is defined as germ cell proliferation rate >10% by 7 days, resulting in at least doubling of germ cell numbers by 21 days with continued proliferation.

### Functional Analysis of GSCs Expanded In Vitro

To investigate functionality of germ cells after expansion in culture, GSCs grown under the best conditions identified above are aggregated 1:2 with freshly obtained testicular somatic cells depleted of germ cells by differential plating or harvested from 1 wk old DAZL^{-/-} boar testes, and grafted under the back skin of castrated nude mice as described^{58,64}. Mice receive 2 aggregates (10×10⁶ cells each) per animal from cultured germ cells aggregated with primary somatic cells, and 2 control aggregates with somatic cells only. Cells are tested from all 3 donor ages, cultured for 7 or 28 days (3 experiment replicates, 72 mice in total). Twelve and 40 wks later, aggregates are recovered (2 animals per collection point) and analyzed for establishment of spermatogenesis (identified by IF as above).

Sequential adhesion culture alone or in combination with bioreactor culture will result in cell populations containing >70% UCH-L1+ germ cells and that yield per gram tissue is similar for all donor ages. Judging from the inventor's previous work and work in rodents it is expected that proliferation of germ cells will improve in low serum or serum replacement culture as serum replacement and culture at below ambient oxygen levels appeared to selectively suppress overgrowth of contaminating somatic cells in preliminary experiments. To ascertain that expansion in vitro did not negatively affect germ cell function, their ability to support spermatogenesis in a xenografting assay that is technically easier, requires fewer cells and reduces the number of large animals needed compared to homologous transplantation to pigs is tested. It is expected that germ cells will still support complete spermatogenesis after 28 days in culture.

### Example 5. Verifying the GST Platform for Germline Rescue and Breeding of DCM Animal Models

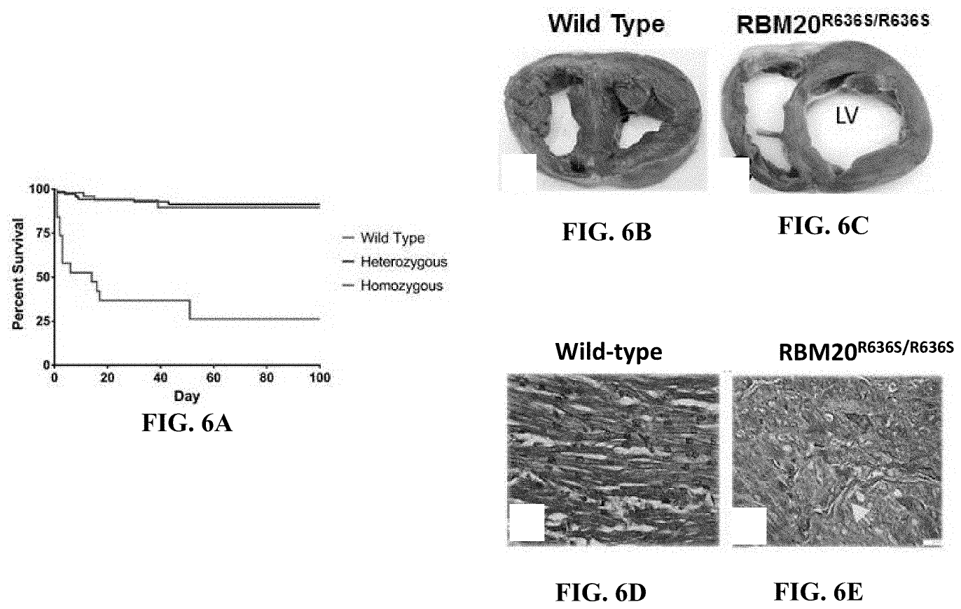

A model of severe pediatric dilated cardiomyopathy (DCM) has been developed by homozygous mutation of the RBM20 gene in Landrace pigs. See, for example PCT/US2017/075270, hereby incorporated by reference in its entirety for all purposes. In the absence of intervention ˜50% of homozygotes die at birth with more dying by 4 weeks of age due to severe heart failure (FIG. 6). Furthermore, no homozygotes have reached sexual maturity, hence are unable to breed due to the severity of the disease/phenotype. The requirement of heterozygous propagation of the RBM20 allele combined with high rates of homozygous, prepubertal mortality results in only ˜10% of piglets reaching the salable age of 4 weeks, significantly increasing production costs. The DCM model is an ideal candidate for the DAZL GST platform and successful application will double production with the same female herd size. Therefore, GSCs from the RBM20 homozygous males are transplanted into DAZL null boars to rescue the line, overcoming the inability to generate homozygous boars and increase the production of saleable animals by 2-fold.

### Transplantation of RBM20 Null GSCs to DAZL Null Boar Recipients

DAZL^{+/-} males and females are bred by artificial insemination to generate DAZL^{-/-} recipient boars. Donor cells are isolated from 3-8 wk old RBM20 homozygotes. Transplantation of 30 million cells to each testes of individual recipients, or the minimal successful dosage is desired. Donor cells from >1 homozygous boars are pooled if necessary for transplant to 3 recipients using the methods described in above. Analysis of DAZL null RBM20 GST recipients. GST boars are analyzed for donor-derived spermatogenesis followed by characterization of spermatogenesis as done in Aim 1.

Artificial insemination with GST-derived sperm and blastocyst analysis. GST ejaculates demonstrating suitable semen quality and characteristics are used for artificial insemination. Large white (Landrace) pigs are inseminated with 2 billion live sperm in 100 ml per insemination. Pregnancy checks are conducted at days 25, 50, and 100. The blastocysts are analyzed for the donor-derived genotype as previously shown⁴⁶. Briefly, individual blastocysts will undergo whole genome amplification, followed by PCR amplification and DNA sequencing.

### Example 6. Evaluate Blastocyst Complementation (BC) for Phenotypic and Germline Rescue of Immunodeficient Lines

Most lineage/organogenesis-deficient null lines are not suitable breeders due to failure to thrive. A T-, B- and NK cell-deficient SCID line (RAG2 and IL2Rg KO) suffers chronic infections that lead to neonatal lethality requiring propagation from heterozygotes that results in only 6.3% of useful embryos for analyses including blastocyst complementation (BC) studies. Although the SCID line can theoretically be rescued after birth using bone-marrow transplantation, similar approaches for the post-birth rescue of organ-deficient lines are currently unavailable. Given the success of BC to restore pancreas in pancreatogenesis-disabled swine and yield fertile adults⁴¹, it is postulated that the same approach can be used to rescue the SCID line. To facilitate the more efficient production of the SCID swine, BC is used to create chimeras of DAZL null and SCID cells. Used as donors, the DAZL null cells can rescue the lethal phenotype, but because the germ cells are absent before puberty, only gametes carrying the lineage/organogenesis-deficient genotype are produced in adults. The successful application of BC in males and females using DAZL null donor cells and subsequent breeding will yield 100% SCID animals and support the expanded utility of this approach for the efficient propagation of other lineage/organogenesis-deficient lines.

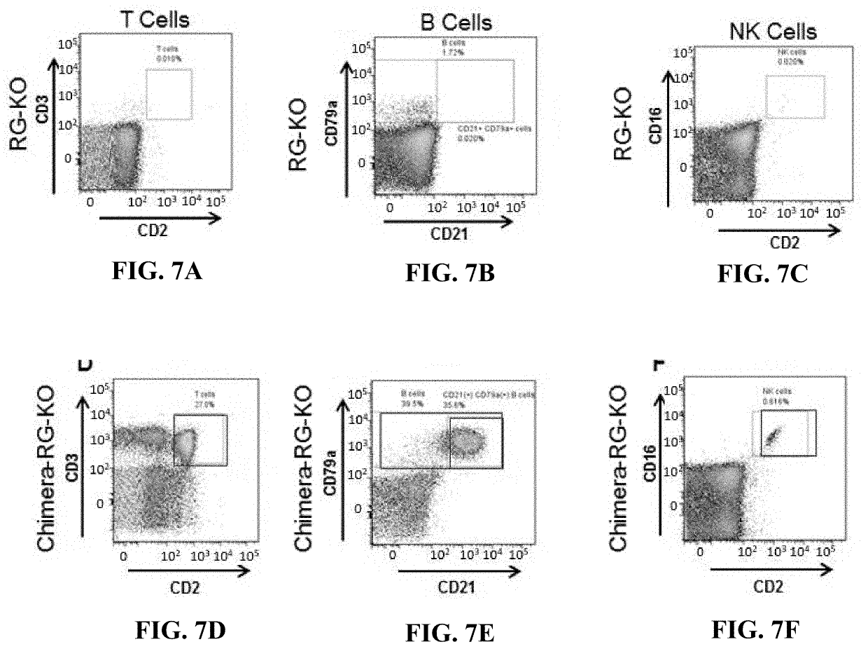

Blastocyst complementation restores lymphocytes in SCID piglets. SCID fibroblasts were produced using TALEN-mediated multiplex knockout of RAG2 and IL2Rg (also referred to as RG-KO). Newborn SCID animals, produced by cloning, lacked thymus and no peripheral or mesentery lymph nodes could be identified (not shown). Analysis of CD45 positive cells from the spleen revealed a complete ablation of T-, B-, and NK-cells (FIG. 7, A-C). To attempt phenotypic rescue, wild-type, EGFP labeled donor blastomeres were injected into SCID blastocysts and transferred to synchronized recipients. Two piglets resulted from one pregnancy. Unlike the non-complemented SCID animals, gross examination of these newborn piglets revealed a normal thymus with readily observable peripheral and mesentery lymph nodes. The SCID genotype was confirmed in these animals, however, the majority of cells in the thymus and spleen were EGFP positive indicating both animals were chimeric (data not shown). Of critical importance, EGFP-positive T-, B-, and NK-cells were present in the chimeric piglets indicating successful restoration of lymphocytes by BC (FIG. 7, D-F). Unlike in DAZL null cells, there is no genetic restriction to prevent the wild-type EGFP donor cells from populating the germline; hence, the germline in these animals is likely mosaic. This problem is solved by using germline ablated DAZL cells as the donors to rescue the immune system and establish a SCID breeding herd.

### DAZL Null Females Fail to Produce Follicles

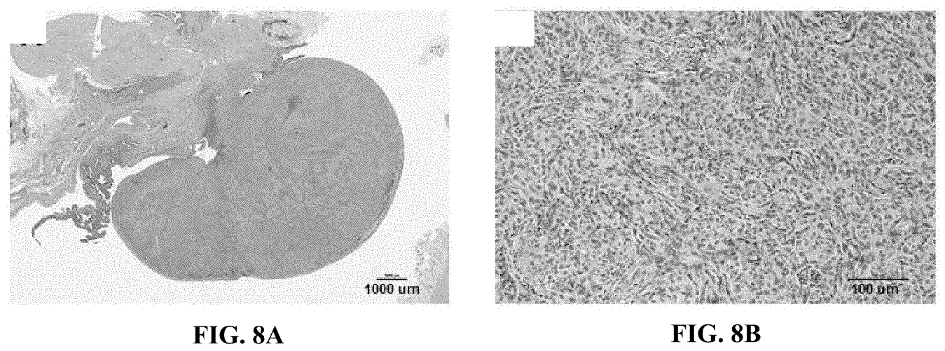

To understand the role of DAZL in female germ cell development, disclosed herein developed DAZL null female swine fibroblasts and generated null females by SCNT. Unlike wild type pigs, the DAZL null females had not exhibited estrus by 1 year of age (wild type typically cycle 6-month age). Necropsy of the animals revealed bilateral abnormality of the ovaries, characterized by a micro ovary, with a diameter at least 3× smaller than a wild type ovary at the same age. No mature or intermediate follicles were present by gross observation. This finding was confirmed by histological analysis (FIG. 8). Consistent with the male phenotype, preliminary staining revealed no germ cells in the micro ovaries, though the time point in which they are lost is unknown due to only sampling adult females. Taken together with the fact that DAZL deficiency is a cell autonomous defect indicates that female chimeras produced with DAZL null donor cells would only produce host gametes.

### Generation of Chimeric Blastocysts Using of DAZL Null Donor and SCID Host Blastocysts

Male and female embryos for BC are prepared using SCNT from fibroblasts from each established line, followed by in vitro culture of embryos to the morula stage. Day 4.5 blastomeres isolated from DAZL null morula are injected into SCID morula at the same stage prior to embryo transfer to a synchronized sow. This approach was successful for chimera production in 2/2 piglets produced in FIG. 7, and a 35% rate of chimera production for Matsunari and colleagues⁴¹. Injected blastocysts are transferred into a total of 16 (8 for male, 8 for female) synchronized recipients in three sessions, each separated by one month. Pregnancy checks are conducted at days 25, 50, and 100. BC is directed by RCI under a contract research agreement.

### Analysis of Phenotypic Rescue and Characterization of Gametogenesis of Chimeric Embryos

At birth, piglets are analyzed for chimerism of cord blood, ear and tail tissues using PCR analysis and an RFLP assay for DAZL null and SCID alleles. Phenotypic rescue is assessed by evaluation of circulating levels of T-, B- and NK cells using fluorescence-activated cell sorting (FACS) shortly after birth. Chimeric piglets, ones with normal levels of T-, B, and NK cells, are reared in standard conditions through sexual maturity. Evaluation of germ cell contribution is performed by hemicastration analysis at 10 weeks of age, followed by histological analysis, as well as GSC isolation to evaluate genotypes of purified germ cells. Semen collection, analysis and characterization of spermatogenesis in chimeric males is performed as discussed above. Chimeric boar fertility is assessed by artificial insemination of wild type or chimeric DAZL females. Fertility in chimeric females is assessed first by observation of for estrus cycling followed by artificial insemination with SCID chimeric male semen. At 1-year chimeric females are sacrificed for characterization of oogenesis using histology and immunohistochemistry. Ovarian tissue is isolated from 3 locations for analysis. Progression of folliculogenesis is characterized by the following germ cell and somatic cell markers using immunohistochemistry: Oocytes—GDF9 and VASA⁴⁰, leptin⁶⁵, androgen receptor⁶⁶. Granulosa cells—Inhibin α⁶⁷, androgen receptor and follicule stimulating hormone receptor⁶⁶.

Restoration of the immune system via BC is established in rodents³⁶. Additionally, pancreas-deficient swine complemented with wildtype cells grew into fertile adults⁴¹, and in previous studies the applicants have successfully complemented T- B- and NK-cells in an SCID host. Historical chimera production rates in pigs range from 20-50%^{41,43}. In non-chimeric pigs, an absence of T, B and NK cells in peripheral blood is expected, but nearly wild-type levels in chimeric pigs. Moreover, it is expected that T, B and NK cell positive chimeras will remain healthy when reared in standard conditions and be fertile.

Previous pig chimera studies have revealed a complex relationship between germline contributions of donor versus host genetics. Factors such as stage of cells from the donor versus stage of the host embryo can result in no donor contribution to 100% donor contribution, though germline contribution was skewed towards the host embryo genotype⁴³. Due to this bias, generally only DAZL null cells are used as donors versus host. However, those of skill in the art will readily appreciate that complementation can occur when DAZL null cells are used as host. Currently, it is known that germ cells are absent in DAZL null boars by 10 weeks of age, and absent in females by 1 year of age; however, if they are not lost early in development there may not be a selective advantage for SCID germ cells. As mentioned above, the use of SCID embryos as hosts will skew towards SCID germline, and residual DAZL null germ cells would be absent by sexual maturity. Regardless of when they are lost, DAZL null animals cannot produce gametes, so it is expected that 100% of gametes from chimeras are SCID-derived by sexual maturity. However, it should be appreciated that the inventors could choose an alternative gene KO that results in germ cell loss at an earlier time point, such as NANOS3⁴⁰. Fertility of chimeric pigs is well documented^{41,43}, but if artificial insemination of SCID chimeric females is not successful with SCID chimeric sperm, wildtype sperm is used to assess fertility. Similarly, fertility of SCID can be assessed on wildtype oocytes.

### Example 7. Successful Application of Germline Stem Cell Transplantation Using Genetically Similar and Divergent Breed GSC Donors

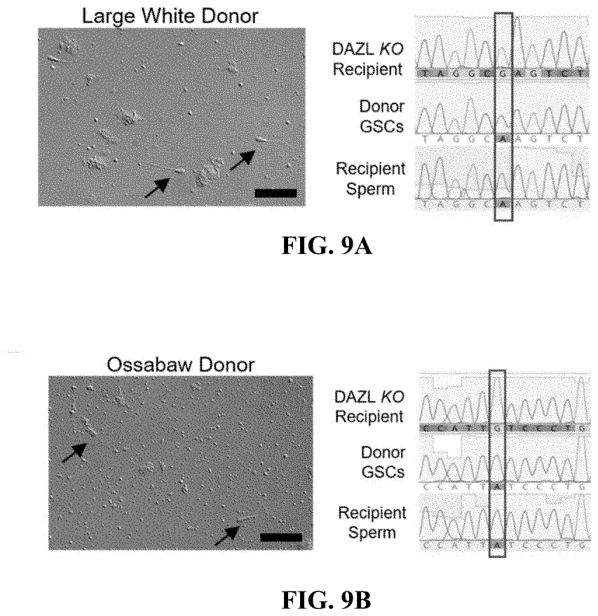

GSCs were isolated from 9 week old Large White (FIG. 9A) or 2 Ossabaw (FIG. 9B). donors were transplanted to one testis of individual 13 week old DAZL KO recipients. Beginning at 6 months of age (sexual maturity) GST recipients were trained for semen collection. Ejaculates were analyzed for the presence of sperm (black arrows) and differentially extracted to reduce the recipient's non-sperm cells within the seminal plasma and concentrate the sperm heads (scale bar 50 um). Single nucleotide polymorphisms (SNP) identified for the recipient tail and donor GSC genomic DNA were PCR amplified and Sanger sequenced. SNP analysis showed transmission of donor-derived sperm in the ejaculates of GST DAZL KO recipients transplanted with Large White (FIG. 9A) or Ossabaw (FIG. 9B) GSCs.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### Certain Embodiments

Embodiment 1 provides a method of producing non-human animal models having congenital defects comprising: i. editing a cell to create one or more genetic lesions/defects in an animal model; ii. cloning the fibroblast or primary cell to provide a first line; iii. creating an embryo from the cell; iv. complementing the genetic defects in the development of the embryo by providing a donor cell that does not comprise the genetic lesion/defects of the first line with the donor cell being gametogenically deficient, to provide a chimera.

Embodiment 2 provides the method of embodiment 1, further comprising, harvesting germline stem cells (GSC) from the chimera; - - - transplanting the GSC from the chimera into the testis of a gametogenically deficient animal wherein the GSC differentiate into sperm or ova; wherein the sperm are used to impregnate a female chimera of claim 1, step iii; wherein the ova are fertilized by the sperm of a male chimera of claim 1, step iii; wherein the resulting progeny have the genotype of the first line are homozygous for the genetic lesions.

Embodiment 3 provides the method of embodiment 1 or 2, and further comprising, breeding a female chimera with a male chimera to provide non-chimeric progeny that are solely of the first line, having congenital defects.

Embodiment 4 provides the method of any one of embodiments 1-3, wherein the animal is a livestock animal.

Embodiment 5 provides the method of any one of embodiments 1-4, wherein the livestock animal is a cattle, pig, goat or sheep.

Embodiment 6 provides the method of any one of embodiments 1-5, wherein the pig is a mini pig.

Embodiment 7 provides the method of any one of embodiments 1-6, wherein the min-pig is selected from Ossabaw, Goettingen, Yucatan, Bama Xiang Zhu, Sinclair, Hanford, Wuzhishan and Xi Shuang Banna.

Embodiment 8 provides the method of any one of embodiments 1-7, wherein the gametogenically deficient animal is a deleted-in-azoospermia-like knockout (DAZL−/−) animal.

Embodiment 9 provides the method of any one of embodiments 1-8, wherein the wherein the genetic lesion is in one or more genes comprising, RNA-Binding Motif Protein 20 (RBM20), Interleukin 2 Receptor Subunit Gamma (IL2Rg), Recombination Activating 2 (RAG2), polycystin-1 (PKD1), polycystin 2 (PKD2), and/or Fibrocystin/Polyductin (PKHD1).

Embodiment 10 provides the method of any one of embodiments 1-10, wherein the non-human animal is heterozygous for the one or more gene edits.

Embodiment 11 provides the method of any one of embodiments 1-10, wherein the non-human animal is homozygous for the one or more gene edits.

Embodiment 12 provides the method of any one of embodiments 1-11, wherein the cell is a primary cell, a fibroblast or a stem cell.

Embodiment 13 provides a method of producing a non-human animal model having congenital defects comprising: i) creating one or more genetic lesions/defects in a first cell to provide a genotype of a first line; ii) providing a second cell that is gametogenically deficient; iii) cloning the first and second cells to provide a first and second embryos; iv) using the first or second embryos as a host and the remaining embryo as a donor; v) transferring one or more cells from the donor embryo and implanting them in the host embryo to create a healthy chimera by complementation of the genetic defects of the first line; vi) wherein the gametes of the chimera have the genotype of the first line; and vii) breeding a male and female of the first line to provide offspring that are non-chimeric and only of the first line.

Embodiment 14 provides the method of embodiment 13, wherein the donor embryo is of the first line.

Embodiment 15 provides the method of any one of embodiments 13-14, wherein the host embryo is of the first line

Embodiment 16 provides the method of any one of embodiments 13-15, wherein the animal is a livestock animal.

Embodiment 17 provides the method of any one of embodiments 13-16, wherein the livestock animal is a cattle, pig, goat or sheep.

Embodiment 18 provides the method of any one of embodiments 13-17, wherein the pig is a mini pig.

Embodiment 19 provides the method of any one of embodiments 13-18, wherein the min-pig is selected from Ossabaw, Goettingen, Yucatan, micro Yucatan, Bama Xiang Zhu, Wuzhishan, Sinclair, Hanford, and Xi Shuang Banna.

Embodiment 20 provides the method of any one of embodiments 13-19, wherein the gametogenically deficient animal is DAZL−/−.

Embodiment 21 provides the method of any one of embodiments 13-20, wherein the genetic lesion comprises one or more genes comprising RNA-Binding Motif Protein 20 (RBM20), Interleukin 2 Receptor Subunit Gamma (IL2Rg), Recombination Activating 2 (RAG2), polycystin-1 (PKD1), polycystin 2 (PKD2), and/or Fibrocystin/Polyductin (PKHD1).

Embodiment 22 provides the method of any one of embodiments 13-21, wherein the animal is heterozygous for one or more gene edits.

Embodiment 23 provides the method of any one of embodiments 13-22, wherein the animal is homozygous for one or more gene edits.

Embodiment 24 provides the method of any one of embodiments 13-23, wherein the first cell is a fibroblast, primary cell or stem cell.

Embodiment 25 provides the method of any one of embodiments 13-24, wherein the second cell is a fibroblast, primary cell of stem cell.

Embodiment 26 provides a method of creating a chimeric blastocyst, morula or embryo for producing animals with a genetic edit that causes a failure to thrive phenotype comprising: obtaining a host blastocyst, morula or embryo from an animal with the genetic edit that causes the failure to thrive phenotype; obtaining a donor cell from a donor animal with a deleted-in-azoospermia like (DAZL) knock out mutation and without the genetic edit that causes the failure to thrive phenotype; and introducing the donor cell to the host blastocyst, morula or embryo to create a chimeric blastocyst, morula or embryo.

Embodiment 27 provides the method of embodiment 26, wherein the failure to thrive phenotype comprises a reduced ability to produce offspring that survive to sexual maturity relative to an animal that does not have the genetic edit that causes the failure to thrive phenotype.

Embodiment 28 provides the method of embodiment 26 or 27, wherein the donor animal does not produce sufficient functional gametes to reproduce.

Embodiment 29 provides the method of any one of embodiments 26-28, wherein the chimeric blastocyst, embryo, or morula is implanted into a surrogate mother to produce an offspring of the animal with the genetic edit that causes the failure to thrive phenotype.

Embodiment 30 provides the method of embodiment 29, wherein the offspring comprises the genetic edit that causes the failure to thrive phenotype.

Embodiment 31 provides the method of embodiment 30, wherein the offspring is heterozygous for the genetic edit that causes the failure to thrive phenotype.

Embodiment 32 provides the method of embodiment 30, wherein the offspring is homozygous for the genetic edit that causes the failure to thrive phenotype.

Embodiment 33 provides the method of any one of embodiments 29-32, wherein the surrogate mother does not comprise the genetic edit that causes the failure to thrive phenotype.

Embodiment 34 provides the method of any one of embodiments 29-33, wherein the offspring does not comprise a genotype of the donor animal.

Embodiment 35 provides the method of any one of embodiments 26-34, wherein the genetic edit that causes the failure to thrive phenotype comprises a genetic edit in a gene selected from the group consisting RNA-Binding Motif Protein 20 (RBM20), Interleukin 2 Receptor Subunit Gamma (IL2Rg), Recombination Activating 2 (RAG2), polycystin-1 (PKD1), polycystin 2 (PKD2), and Fibrocystin/Polyductin (PKHD1).

Embodiment 36 provides the method of any one of embodiments 26-35, wherein the animal with the genetic edit that causes the failure to thrive phenotype or the donor animal or the animal with the genetic edit that causes the failure to thrive phenotype and the donor animal is a livestock animal.

Embodiment 37 provides the method of embodiment 36, wherein the livestock animal is selected from the group consisting of cattle, pig, goat, and sheep.

Embodiment 38 provides the method of embodiment 37, wherein the pig is a mini-pig.

Embodiment 39 provides the method of embodiment 38, wherein the mini-pig is selected from the group consisting of Ossabaw, Goettingen, Yucatan, Bama Xiang Zhu, Wuzhishan and Xi Shuang Banna.

Embodiment 40 provides the method of any one of embodiments 26-39, wherein the donor cell is a stem cell.

Embodiment 41 provides the method of any one of embodiments 26, and 28-40, wherein the failure to thrive phenotype comprises a reduced ability to grow or a reduced ability to reach maturity relative to an animal that does not have the genetic edit that causes the failure to thrive phenotype.

Embodiment 42 provides the method of any one of embodiments 26, and 28-40, wherein the failure to thrive phenotype comprises a lineage deficiency phenotype or an organogenesis deficiency phenotype.

Embodiment 43 provides a method for producing animals with a genetic edit that causes a failure to thrive phenotype comprising: obtaining a cell of an animal that does not have the genetic edit that causes the failure to thrive phenotype; editing a gene of the cell of the animal that does not have the genetic edit that causes the failure to thrive phenotype in a manner to cause a second animal created from the cell of the first animal with the edited gene to have the genetic edit that causes the failure to thrive phenotype; creating a host blastocyst, morula or embryo from the cell with the edited gene; obtaining one or more donor cells from a donor animal, with the one or more donor cells having a deleted-in-azoospermia like (DAZL) knock out mutation and not having the genetic edit that causes the failure to thrive phenotype; introducing the one or more donor cells to the host blastocyst, morula or embryo to create a chimeric blastocyst, morula or embryo; allowing the chimeric blastocyst to develop; and with the developed chimeric blastocyst, morula or embryo or cells therefrom, producing animals with the genetic edit that causes the failure to thrive phenotype.

Embodiment 44 provides the method of embodiment 43, wherein the failure to thrive phenotype comprises a reduced ability to produce offspring that survive to sexual maturity relative to an animal that does not have the genetic edit that causes the failure to thrive phenotype.

Embodiment 45 provides the method of embodiment 43, wherein the failure to thrive phenotype comprises a reduced ability to grow or a reduced ability to reach maturity relative to an animal that does not have the genetic edit that causes the failure to thrive phenotype.

Embodiment 46 provides the method of method of embodiment 43, wherein the failure to thrive phenotype comprises a lineage deficiency phenotype or an organogenesis deficiency phenotype.

Embodiment 47 provides the methods of any one of embodiments 43-46, wherein producing animals with the genetic edit that causes the failure to thrive phenotype comprises producing animals that are heterozygous for the genetic edit that causes the failure to thrive phenotype.

Embodiment 48 provides the methods of any one of embodiments 43-46, wherein producing animals with the genetic edit that causes the failure to thrive phenotype comprises producing animals that are homozygous for the genetic edit that causes the failure to thrive phenotype.

Embodiment 49 provides the method of any one of embodiments 43-48, wherein the genetic edit that causes the failure to thrive phenotype comprises a genetic edit in a gene selected from the group consisting RNA-Binding Motif Protein 20 (RBM20), Interleukin 2 Receptor Subunit Gamma (IL2Rg), Recombination Activating 2 (RAG2), polycystin-1 (PKD1), polycystin 2 (PKD2), and Fibrocystin/Polyductin (PKHD1).

Embodiment 50 provides a method of breeding an animal with a genetic edit that causes a failure to thrive phenotype comprising obtaining a host blastocyst, embryo, or morula from the animal with the genetic edit that causes the failure to thrive phenotype and introducing to the host blastocyst, embryo, or morula, a donor cell from a donor animal that comprises a deleted-in-azoospermia like (DAZL) knock out mutation and does not comprise the genetic edit that causes the failure to thrive phenotype to create a chimeric blastocyst, embryo, or morula.

Embodiment 51 provides the method of embodiment 50, wherein the failure to thrive phenotype comprises a reduced ability to produce offspring that survive to sexual maturity relative to an animal that does not have the genetic edit that causes the failure to thrive phenotype.

Embodiment 52 provides the method of embodiment 50, wherein the failure to thrive phenotype comprises a reduced ability to grow or a reduced ability to reach maturity relative to an animal that does not have the genetic edit that causes the failure to thrive phenotype.

Embodiment 53 provides the method of embodiment 50, wherein the failure to thrive phenotype comprises a lineage deficiency phenotype or an organogenesis deficiency phenotype.

Embodiment 54 provides the method of any one of embodiments 50-53, wherein the donor animal does not produce sufficient functional gametes to reproduce.

Embodiment 55 provides the method of any one of embodiments 50-54, wherein the chimeric blastocyst, embryo, or morula is implanted into a surrogate mother to produce an offspring of the animal with the genetic edit that causes the failure to thrive phenotype.

Embodiment 56 provides the method of embodiment 55, wherein the offspring comprises the genetic edit that causes the failure to thrive phenotype.

Embodiment 57 provides the method of embodiment 56, wherein the offspring is heterozygous for the genetic edit that causes the failure to thrive phenotype.

Embodiment 58 provides the method of embodiment 56, wherein the offspring is homozygous for the genetic edit that causes the failure to thrive phenotype.

Embodiment 59 provides the method of any one of embodiments 55-58, wherein the surrogate mother does not comprise the genetic edit that causes the failure to thrive phenotype.

Embodiment 60 provides the method of any one of embodiments 55-59, wherein the offspring does not comprise a genotype of the donor animal.

Embodiment 61 provides the method of any one of embodiments 50-60, wherein the genetic edit that causes the failure to thrive phenotype comprises a genetic edit in a gene selected from the group consisting RNA-Binding Motif Protein 20 (RBM20), Interleukin 2 Receptor Subunit Gamma (IL2Rg), Recombination Activating 2 (RAG2), polycystin-1 (PKD1), polycystin 2 (PKD2), and Fibrocystin/Polyductin (PKHD1).

Embodiment 62 provides the method of any one of embodiments 50-61, wherein the animal with the genetic edit that causes the failure to thrive phenotype or the donor animal or the animal with the genetic edit that causes the failure to thrive phenotype and the donor animal is a livestock animal.

Embodiment 63 provides the method of embodiment 62, wherein the livestock animal is selected from the group consisting of cattle, pig, goat, and sheep.

Embodiment 64 provides the method of embodiment 63, wherein the pig is a mini-pig.

Embodiment 65 provides the method of embodiment 64, wherein the mini-pig is selected from the group consisting of Ossabaw, Goettingen, Yucatan, Bama Xiang Zhu, Wuzhishan and Xi Shuang Banna.

Embodiment 66 provides the method of any one of embodiments 50-65, wherein the donor cell is a stem cell.

Embodiment 67 provides a chimeric blastocyst, embryo, or morula comprising a host blastocyst, embryo, or morula from an animal with a genetic edit that causes a failure to thrive phenotype and a donor cell from a donor animal with a DAZL knock out mutation and without the genetic edit that causes the failure to thrive phenotype.

Embodiment 68 provides the chimeric blastocyst, embryo, or morula of embodiment 67, wherein the failure to thrive phenotype comprises a reduced ability to produce offspring that survive to sexual maturity relative to an animal that does not have the genetic edit that causes the failure to thrive phenotype.

Embodiment 69 provides the chimeric blastocyst, embryo, or morula of embodiment 67, wherein the failure to thrive phenotype comprises a reduced ability to grow or a reduced ability to reach maturity relative to an animal that does not have the genetic edit that causes the failure to thrive phenotype.

Embodiment 70 provides the chimeric blastocyst, embryo, or morula of embodiment 67, wherein the failure to thrive phenotype comprises a lineage deficiency phenotype or an organogenesis deficiency phenotype.

Embodiment 71 provides the chimeric blastocyst, embryo, or morula of any one of embodiments 67-70, wherein the donor animal does not produce sufficient functional gametes to reproduce.

Embodiment 72 provides the chimeric blastocyst, embryo, or morula of any one of embodiments 67-71, wherein the genetic edit that causes the failure to thrive phenotype comprises a genetic edit in a gene selected from the group consisting RNA-Binding Motif Protein 20 (RBM20), Interleukin 2 Receptor Subunit Gamma (IL2Rg), Recombination Activating 2 (RAG2), polycystin-1 (PKD1), polycystin 2 (PKD2), and Fibrocystin/Polyductin (PKHD1).

Embodiment 73 provides the chimeric blastocyst, embryo, or morula of any one of embodiments 67-72, wherein the animal with the genetic edit that causes the failure to thrive phenotype or the donor animal or the animal with the genetic edit that causes the failure to thrive phenotype and the donor animal is a livestock animal.

Embodiment 74 provides the chimeric blastocyst, embryo, or morula of embodiment 73, wherein the livestock animal is selected from the group consisting of cattle, pig, goat, and sheep.

Embodiment 75 provides the chimeric blastocyst, embryo, or morula of embodiment 74, wherein the pig is a mini-pig.

Embodiment 76 provides the chimeric blastocyst, embryo, or morula of embodiment 75, wherein the mini-pig is selected from the group consisting of Ossabaw, Goettingen, Yucatan, Bama Xiang Zhu, Wuzhishan and Xi Shuang Banna.

Embodiment 77 provides the chimeric blastocyst, embryo, or morula of any one of embodiments 67-76, wherein the donor cell is a stem cell.

Embodiment 78 provides a surrogate mother comprising an implanted chimeric blastocyst, embryo, or morula wherein the chimeric blastocyst, embryo, or morula comprises a host blastocyst, embryo, or morula from an animal with a genetic edit that causes a failure to thrive phenotype and a donor cell from a donor animal with a deleted-in-azoospermia like (DAZL) knock out mutation and without the mutation that causes the failure to thrive phenotype.

Embodiment 79 provides the surrogate mother of embodiment 78, wherein the failure to thrive phenotype comprises a reduced ability to produce offspring that survive to sexual maturity relative to an animal that does not have the genetic edit that causes the failure to thrive phenotype.

Embodiment 80 provides the surrogate mother of embodiment 78, wherein the failure to thrive phenotype comprises a reduced ability to grow or a reduced ability to reach maturity relative to an animal that does not have the genetic edit that causes the failure to thrive phenotype.

Embodiment 81 provides the surrogate mother of embodiment 78, wherein the failure to thrive phenotype comprises a lineage deficiency phenotype or an organogenesis deficiency phenotype.

Embodiment 82 provides the surrogate mother of any one of embodiments 78-81, wherein the donor animal does not produce sufficient functional gametes to reproduce.

Embodiment 83 provides the surrogate mother of any one of embodiments 78-82, wherein the genetic edit that causes the failure to thrive phenotype comprises a genetic edit in a gene selected from the group consisting RNA-Binding Motif Protein 20 (RBM20), Interleukin 2 Receptor Subunit Gamma (IL2Rg), Recombination Activating 2 (RAG2), polycystin-1 (PKD1), polycystin 2 (PKD2), and Fibrocystin/Polyductin (PKHD1).

Embodiment 84 provides the surrogate mother of any one of embodiments 78-83, wherein the animal with the genetic edit that causes the failure to thrive phenotype or the donor animal or the animal with the genetic edit that causes the failure to thrive phenotype and the donor animal is a livestock animal.

Embodiment 85 provides the surrogate mother of embodiment 84, wherein the livestock animal is selected from the group consisting of cattle, pig, goat, and sheep.

Embodiment 86 provides the surrogate mother of embodiment 85, wherein the pig is a mini-pig.

Embodiment 87 provides the surrogate mother of embodiment 86, wherein the mini-pig is selected from the group consisting of Ossabaw, Goettingen, Yucatan, Bama Xiang Zhu, Wuzhishan and Xi Shuang Banna.

Embodiment 88 provides the surrogate mother of any one of embodiments 78-87, wherein the donor cell is a stem cell.

Embodiment 89 provides the surrogate mother of any one of embodiments 78-88, wherein the surrogate mother is a livestock animal.

Embodiment 90 provides the surrogate mother of embodiment 89, wherein the livestock animal is selected from the group consisting of cattle, pig, goat, and sheep.

Embodiment 91 provides the surrogate mother of embodiment 90, wherein the pig is a mini-pig.

Embodiment 92 provides the surrogate mother of embodiment 91, wherein the mini-pig is selected from the group consisting of Ossabaw, Goettingen, Yucatan, Bama Xiang Zhu, Wuzhishan and Xi Shuang Banna.

Embodiment 93 provides the surrogate mother of any one of embodiments 78-92, wherein the surrogate mother does not comprise the genetic edit that causes the failure to thrive phenotype.

Embodiment 94 provides the animal produced from the implanted chimeric blastocyst, embryo, or morula of any one of embodiments 78-93.

Embodiment 95 provides the progeny of the animal of embodiment 94.

Embodiment 96 provides a method of breeding an animal with a genetic edit that causes a failure to thrive phenotype comprising introducing a germline stem cell from the animal with the genetic edit that causes the failure to thrive phenotype to a testis of a host animal that comprises a deleted-in-azoospermia like (DAZL) knock out mutation and that does not comprise the genetic edit that causes the failure to thrive phenotype wherein the germline stem cell introduced to the testis matures to produce a functional sperm comprising the genetic edit that causes the failure to thrive phenotype.

Embodiment 97 provides the method of embodiment 96, wherein the failure to thrive phenotype comprises a reduced ability to produce offspring that survive to sexual maturity relative to an animal that does not have the genetic edit that causes the failure to thrive phenotype.

Embodiment 98 provides the method of embodiment 96, wherein the failure to thrive phenotype comprises a reduced ability to grow or a reduced ability to reach maturity relative to an animal that does not have the genetic edit that causes the failure to thrive phenotype.

Embodiment 99 provides the method of embodiment 96, wherein the failure to thrive phenotype comprises a lineage deficiency phenotype or an organogenesis deficiency phenotype.

Embodiment 100 provides the method of any one of embodiments 96-99, wherein the functional sperm comprising the genetic edit that causes the failure to thrive phenotype is used to fertilize a donor ovum to produce an embryo.

Embodiment 101 provides the method of embodiment 100, wherein the donor ovum is heterozygous for the genetic edit that causes the failure to thrive phenotype.

Embodiment 102 provides the method of embodiment 100, wherein the donor ovum does not comprise the genetic edit that causes the failure to thrive phenotype.

Embodiment 103 provides the method of any one of embodiments 100-102, wherein the embryo is implanted into a surrogate mother to produce an offspring comprising the genetic edit that causes the failure to thrive phenotype.

Embodiment 104 provides the method of embodiment 103, wherein the offspring is heterozygous for the genetic edit that causes the failure to thrive phenotype.

Embodiment 105 provides the method of embodiment 103, wherein the offspring is homozygous for the genetic edit that causes the failure to thrive phenotype.

Embodiment 106 provides the method of any one of embodiments 103-105, wherein the offspring does not comprise a genotype of the host animal that comprises the DAZL knock out mutation.

Embodiment 107 provides the method of any one of embodiments 96-106, wherein the genetic edit that causes the failure to thrive phenotype comprises a genetic edit in a gene selected from the group consisting RNA-Binding Motif Protein 20 (RBM20), Interleukin 2 Receptor Subunit Gamma (IL2Rg), Recombination Activating 2 (RAG2), polycystin-1 (PKD1), polycystin 2 (PKD2), and Fibrocystin/Polyductin (PKHD1).

Embodiment 108 provides the method of any one of embodiments 96-107, wherein the animal with the genetic edit that causes the failure to thrive phenotype is a livestock animal.

Embodiment 109 provides the method of embodiment 108, wherein the livestock animal is selected from the group consisting of cattle, pig, goat, and sheep.

Embodiment 110 provides the method of embodiment 109, wherein the pig is a mini-pig.

Embodiment 111 provides the method of embodiment 110, wherein the mini-pig is selected from the group consisting of Ossabaw, Goettingen, Yucatan, Bama Xiang Zhu, Wuzhishan and Xi Shuang Banna.

Embodiment 112 provides the method of any one of embodiments 96-111, wherein the host animal that comprises the DAZL knock mutation is a livestock animal.

Embodiment 113 provides the method of embodiment 112, wherein the livestock animal is selected from the group consisting of cattle, pig, goat, and sheep.

Embodiment 114 provides the method of embodiment 113, wherein the pig is a mini-pig.

Embodiment 115 provides the method of embodiment 114, wherein the mini-pig is selected from the group consisting of Ossabaw, Goettingen, Yucatan, Bama Xiang Zhu, Wuzhishan and Xi Shuang Banna.

Embodiment 116 provides the method of any one of embodiments 100-115, wherein the donor ovum is from an animal that is a livestock animal.

Embodiment 117 provides the method of embodiment 116, wherein the livestock animal is selected from the group consisting of cattle, pig, goat, and sheep.

Embodiment 118 provides the method of embodiment 117, wherein the pig is a mini-pig.

Embodiment 119 provides the method of embodiment 118, wherein the mini-pig is selected from the group consisting of Ossabaw, Goettingen, Yucatan, Bama Xiang Zhu, Wuzhishan and Xi Shuang Banna.

Embodiment 120 provides the method of any one of embodiments 103-119, wherein the surrogate mother is a livestock animal.

Embodiment 121 provides the method of embodiment 120, wherein the livestock animal is selected from the group consisting of cattle, pig, goat, and sheep.

Embodiment 122 provides the method of embodiment 121, wherein the pig is a mini-pig.

Embodiment 123 provides the method of embodiment 122, wherein the mini-pig is selected from the group consisting of Ossabaw, Goettingen, Yucatan, Bama Xiang Zhu, Wuzhishan and Xi Shuang Banna.

Embodiment 124 provides a host animal for breeding an animal with a genetic edit that causes a failure to thrive, the host animal comprising a genome with a deleted-in-azoospermia like (DAZL) knock out mutation and that does not comprise the genetic edit that causes the failure to thrive mutation and wherein the host animal comprises a testis containing a transplanted germline stem cell from an animal with the genetic edit that causes the failure to thrive phenotype.

Embodiment 125 provides the host animal of embodiment 124, wherein the failure to thrive phenotype comprises a reduced ability to produce offspring that survive to sexual maturity relative to an animal that does not have the genetic edit that causes the failure to thrive phenotype.

Embodiment 126 provides the host animal of embodiment 124, wherein the failure to thrive phenotype comprises a reduced ability to grow or a reduced ability to reach maturity relative to an animal that does not have the genetic edit that causes the failure to thrive phenotype.

Embodiment 127 provides the host animal of embodiment 124, wherein the failure to thrive phenotype comprises a lineage deficiency phenotype or an organogenesis deficiency phenotype.

Embodiment 128 provides the host animal of any one of embodiments 124-127, wherein the germline stem cell matures to produce a functional sperm comprising the genetic edit that causes the failure to thrive phenotype.

Embodiment 129 provides the host animal of any one of embodiments 124-128, wherein the genetic edit that causes the failure to thrive phenotype comprises a genetic edit in a gene selected from the group consisting RNA-Binding Motif Protein 20 (RBM20), Interleukin 2 Receptor Subunit Gamma (IL2Rg), Recombination Activating 2 (RAG2), polycystin-1 (PKD1), polycystin 2 (PKD2), and Fibrocystin/Polyductin (PKHD1).

Embodiment 130 provides the host animal of any one of embodiments 124-129, wherein the host animal is a livestock animal.

Embodiment 131 provides the host animal of embodiment 130, wherein the livestock animal is selected from the group consisting of cattle, pig, goat, and sheep.

Embodiment 132 provides the host animal of embodiment 131, wherein the pig is a mini-pig.

Embodiment 133 provides the host animal of embodiment 132, wherein the mini-pig is selected from the group consisting of Ossabaw, Goettingen, Yucatan, Bama Xiang Zhu, Wuzhishan and Xi Shuang Banna.

Embodiment 134 provides the host animal of any one of embodiments 124-133, wherein the animal with the genetic edit that causes the failure to thrive phenotype is a livestock animal.

Embodiment 135 provides the host animal of embodiment 134, wherein the livestock animal is selected from the group consisting of cattle, pig, goat, and sheep.

Embodiment 136 provides the host animal of embodiment 135, wherein the pig is a mini-pig.

Embodiment 137 provides the host animal of embodiment 136, wherein the mini-pig is selected from the group consisting of Ossabaw, Goettingen, Yucatan, Bama Xiang Zhu, Wuzhishan and Xi Shuang Banna.

All patents, publications, and journal articles set forth herein are hereby incorporated by reference herein; in case of conflict, the instant specification is controlling.

While the disclosure has been described in conjunction with the various exemplary embodiments outlined above, various alternatives, modifications, variations, improvements, and/or substantial equivalents, whether known or that are or may be presently unforeseen, may become apparent to those having at least ordinary skill in the art. Accordingly, the exemplary embodiments according to the disclosure, as set forth above, are intended to be illustrative, not limiting. Therefore, the disclosure is intended to embrace all known or later-developed alternatives, modifications, variations, improvements, and/or substantial equivalents of these exemplary embodiments.

### REFERENCES

1 Flisikowska, T., Kind, A. & Schnieke, A. The new pig on the block: modelling cancer in pigs. Transgenic Res 22, 673-680, doi:10.1007/s11248-013-9720-9 (2013).
2 Tan, W. S., Carlson, D. F., Walton, M. W., Fahrenkrug, S. C. & Hackett, P. B. Precision editing of large animal genomes. Advances in genetics 80, 37-97, doi:10.1016/B978-0-12-404742-6.00002-8 (2012).
3 Tang, L., Gonzalez, R. & Dobrinski, I. Germline modification of domestic animals. Anim Reprod 12, 93-104 (2015).
4 Campbell, K. H. Nuclear transfer in farm animal species. Seminars in cell & developmental biology 10, 245-252, doi:10.1006/scdb.1999.0310 (1999).
5 Gama Sosa, M. A., De Gasperi, R. & Elder, G. A. Animal transgenesis: an overview. Brain structure & function 214, 91-109, doi:10.1007/s00429-009-0230-8 (2010).
6 Piedrahita, J. A. & Olby, N. Perspectives on transgenic livestock in agriculture and biomedicine: an update. Reproduction, fertility, and development 23, 56-63, doi:10.1071/RD10246 (2011).
7 Armstrong, L., Lako, M., Dean, W. & Stojkovic, M. Epigenetic modification is central to genome reprogramming in somatic cell nuclear transfer. Stem Cells 24, 805-814, doi:10.1634/stemcells.2005-0350 (2006).
8 Bonk, A. J. et al. Aberrant DNA methylation in porcine in vitro-, parthenogenetic-, and somatic cell nuclear transfer-produced blastocysts. Mol Reprod Dev 75, 250-264, doi:10.1002/mrd.20786 (2008).
9 Niu, D. et al. Inactivation of porcine endogenous retrovirus in pigs using CRISPR-Cas9. Science 357, 1303-1307, doi:10.1126/science.aan4187 (2017).
10 Jefferies, J. L. & Towbin, J. A. Dilated cardiomyopathy. Lancet 375, 752-762, doi:10.1016/S0140-6736(09)62023-7 (2010).
11 Russell, L. D., Ettlin, R., Sinha Kikm, A., Clegg, E. Histological and Histopathological Evaluation of the Testis. (Cache River Press, Clearwater Fl., 1990).
12 Brinster, R. L. & Avarbock, M. R. Germline transmission of donor haplotype following spermatogonial transplantation. Proceedings of the National Academy of Sciences of the United States of America 91, 11303-11307 (1994).
13 Brinster, R. L. & Zimmermann, J. W. Spermatogenesis following male germ-cell transplantation. Proceedings of the National Academy of Sciences of the United States of America 91, 11298-11302 (1994).
14 Honaramooz, A. et al. Fertility and germline transmission of donor haplotype following germ cell transplantation in immunocompetent goats. Biology of reproduction 69, 1260-1264, doi:10.1095/biolreprod.103.018788 (2003).
15 Honaramooz, A., Megee, S. O. & Dobrinski, I. Germ cell transplantation in pigs. Biology of reproduction 66, 21-28 (2002).
16 Herrid, M. et al. Irradiation enhances the efficiency of testicular germ cell transplantation in sheep. Biology of reproduction 81, 898-905, doi:10.1095/biolreprod.109.078279 (2009).
17 Izadyar, F. et al. Autologous and homologous transplantation of bovine spermatogonial stem cells. Reproduction 126, 765-774 (2003).
18 Honaramooz, A. & Yang, Y. Recent advances in application of male germ cell transplantation in farm animals. Vet Med Int 2011, doi:10.4061/2011/657860 (2010).
19 Ogawa, T., Dobrinski, I. & Brinster, R. L. Recipient preparation is critical for spermatogonial transplantation in the rat. Tissue Cell 31, 461-472, doi:10.1054/tice.1999.0060 (1999).
20 Joerg, H. et al. Germ cell transplantation in an azoospermic Klinefelter bull. Biology of reproduction 69, 1940-1944, doi:10.1095/biolreprod.103.020297 (2003).
21 Shinohara, T., Orwig, K. E., Avarbock, M. R. & Brinster, R. L. Remodeling of the postnatal mouse testis is accompanied by dramatic changes in stem cell number and niche accessibility. Proceedings of the National Academy of Sciences of the United States of America 98, 6186-6191, doi:10.1073/pnas.111158198 (2001).
22 Shinohara, T., Orwig, K. E., Avarbock, M. R. & Brinster, R. L. Germ Line Stem Cell Competition in Postnatal Mouse Testes 1. Biology of Reproduction 66, 1491-1497, doi:10.1095/biolreprod66.5.1491 (2002).
23 Honaramooz, A. et al. Depletion of endogenous germ cells in male pigs and goats in preparation for germ cell transplantation. Journal of andrology 26, 698-705, doi:10.2164/jandro1.05032 (2005).
24 Mikkola, M. et al. Transplantation of normal boar testicular cells resulted in complete focal spermatogenesis in a boar affected by the immotile short-tail sperm defect. Reproduction in domestic animals=Zuchthygiene 41, 124-128, doi:10.1111/j.1439-0531.2006.00651.x (2006).
25 Lin, Z. et al. Effective production of recipient male pigs for spermatogonial stem cell transplantation by intratesticular injection with busulfan. Theriogenology 89, 365-373 e362, doi:10.1016/j.theriogenology.2016.10.021 (2017).
26 Rilianawati, Speed, R., Taggart, M. & Cooke, H. J. Spermatogenesis in testes of Dazl null mice after transplantation of wild-type germ cells. Reproduction 126, 599-604 (2003).
27 Ganguli, N. et al. An efficient method for generating a germ cell depleted animal model for studies related to spermatogonial stem cell transplantation. Stem Cell Res Ther 7, 142, doi:10.1186/s13287-016-0405-1 (2016).
28 Ogawa, T., Dobrinski, I., Avarbock, M. R. & Brinster, R. L. Transplantation of male germ line stem cells restores fertility in infertile mice. Nat Med 6, 29-34, doi:10.1038/71496 (2000).
29 Richardson, T. E., Chapman, K. M., Tenenhaus Dann, C., Hammer, R. E. & Hamra, F. K. Sterile testis complementation with spermatogonial lines restores fertility to DAZL-deficient rats and maximizes donor germline transmission. PLoS One 4, e6308, doi:10.1371/journal.pone.0006308 (2009).
30 Park, K. E. et al. Generation of germline ablated male pigs by CRISPR/Cas9 editing of the NANOS2 gene. Sci Rep 7, 40176, doi:10.1038/srep40176 (2017).
31 Luo, J., Megee, S., Rathi, R. & Dobrinski, I. Protein gene product 9.5 is a spermatogonia-specific marker in the pig testis: application to enrichment and culture of porcine spermatogonia. Mol Reprod Dev 73, 1531-1540, doi:10.1002/mrd.20529 (2006).
32 Dores, C., Rancourt, D. & Dobrinski, I. Stirred suspension bioreactors as a novel method to enrich germ cells from pre-pubertal pig testis. Andrology 3, 590-597, doi:10.1111/andr.12031 (2015).
33 Nagano, M., Avarbock, M. R., Leonida, E. B., Brinster, C. J. & Brinster, R. L. Culture of mouse spermatogonial stem cells. Tissue Cell 30, 389-397 (1998).
34 Kubota, H., Avarbock, M. R. & Brinster, R. L. Growth factors essential for self-renewal and expansion of mouse spermatogonial stem cells. Proc Natl Acad Sci USA 101, 16489-16494, doi:10.1073/pnas.0407063101 (2004).
35 Wu, J. & Izpisua Belmonte, J. C. Interspecies chimeric complementation for the generation of functional human tissues and organs in large animal hosts. Transgenic Res 25, 375-384, doi:10.1007/s11248-016-9930-z (2016).
36 Chen, J., Lansford, R., Stewart, V., Young, F. & Alt, F. W. RAG-2-deficient blastocyst complementation: an assay of gene function in lymphocyte development. Proc Natl Acad Sci USA 90, 4528-4532 (1993).
37 Kobayashi, T. et al. Generation of rat pancreas in mouse by interspecific blastocyst injection of pluripotent stem cells. Cell 142, 787-799, doi:10.1016/j.cell.2010.07.039 (2010).
38 Usui, J. et al. Generation of kidney from pluripotent stem cells via blastocyst complementation. Am J Pathol 180, 2417-2426, doi:10.1016/j.ajpath.2012.03.007 (2012).
39 Bort, R., Signore, M., Tremblay, K., Martinez Barbera, J. P. & Zaret, K. S. Hex homeobox gene controls the transition of the endoderm to a pseudostratified, cell emergent epithelium for liver bud development. Dev Biol 290, 44-56, doi:10.1016/j.ydbio.2005.11.006 (2006).
40 Ideta, A. et al. Generation of exogenous germ cells in the ovaries of sterile NANOS3-null beef cattle. Sci Rep 6, 24983, doi:10.1038/srep24983 (2016).
41 Matsunari, H. et al. Blastocyst complementation generates exogenic pancreas in vivo in apancreatic cloned pigs. Proc Natl Acad Sci USA 110, 4557-4562, doi:10.1073/pnas.1222902110 (2013).
42 Wu, J. et al. Interspecies Chimerism with Mammalian Pluripotent Stem Cells. Cell 168, 473-486 e415, doi:10.1016/j.cell.2016.12.036 (2017).
43 Nagashima, H., Giannakis, C., Ashman, R. J. & Nottle, M. B. Sex differentiation and germ cell production in chimeric pigs produced by inner cell mass injection into blastocysts. Biol Reprod 70, 702-707, doi:10.1095/biolreprod.103.022681 (2004).
44 Rogers, C. S. et al. Disruption of the CFTR gene produces a model of cystic fibrosis in newborn pigs. Science 321, 1837-1841, doi:10.1126/science.1163600 (2008).
45 Sommer, J. R. et al. Production of ELOVL4 transgenic pigs: a large animal model for Stargardt-like macular degeneration. Br J Ophthalmol 95, 1749-1754, doi:10.1136/bjophthalmol-2011-300417 (2011).
46 Carlson, D. F. et al. Efficient TALEN-mediated gene knockout in livestock. Proceedings of the National Academy of Sciences of the United States of America 109, 17382-17387, doi:10.1073/pnas.1211446109 (2012).
47 Hickey, R. D. et al. Efficient production of Fah-null heterozygote pigs by chimeric adeno-associated virus-mediated gene knockout and somatic cell nuclear transfer. Hepatology 54, 1351-1359, doi:10.1002/hep.24490 (2011).
48 Tan, W. et al. Efficient nonmeiotic allele introgression in livestock using custom endonucleases. Proc Natl Acad Sci USA 110, 16526-16531, doi:10.1073/pnas.1310478110 (2013).
49 Lai, L. et al. Production of alpha-1,3-galactosyltransferase knockout pigs by nuclear transfer cloning. Science 295, 1089-1092, doi:10.1126/science.1068228 (2002).
50 Ruggiu, M. et al. The mouse Dazla gene encodes a cytoplasmic protein essential for gametogenesis. Nature 389, 73-77, doi:10.1038/37987 (1997).
51 Griswold, M. D. The central role of Sertoli cells in spermatogenesis. Semin Cell Dev Biol 9, 411-416, doi:10.1006/scdb.1998.0203 (1998).
52 Wang, Z. Q., Watanabe, Y., Toki, A. & Itano, T. Altered distribution of Sertoli cell vimentin and increased apoptosis in cryptorchid rats. J Pediatr Surg 37, 648-652 (2002).
53 Zeng, W. et al. Viral transduction of male germline stem cells results in transgene transmission after germ cell transplantation in pigs. Biol Reprod 88, 27, doi:10.1095/biolreprod.112.104422 (2013).
54 Nagano, M. C. Homing efficiency and proliferation kinetics of male germ line stem cells following transplantation in mice. Biol Reprod 69, 701-707, doi:10.1095/biolreprod.103.016352 (2003).
55 Zohni, K., Zhang, X., Tan, S. L., Chan, P. & Nagano, M. C. The efficiency of male fertility restoration is dependent on the recovery kinetics of spermatogonial stem cells after cytotoxic treatment with busulfan in mice. Hum Reprod 27, 44-53, doi:10.1093/humrep/der357 (2012).
56 Luo, J., Megee, S. & Dobrinski, I. Asymmetric distribution of UCH-L1 in spermatogonia is associated with maintenance and differentiation of spermatogonial stem cells. J Cell Physiol 220, 460-468, doi:10.1002/jcp.21789 (2009).
57 Zeng, W. et al. Lymphoid-specific helicase (HELLS) is essential for meiotic progression in mouse spermatocytes. Biology of reproduction 84, 1235-1241, doi:10.1095/biolreprod.110.085720 (2011).
58 Honaramooz, A., Megee, S. O., Rathi, R. & Dobrinski, I. Building a testis: formation of functional testis tissue after transplantation of isolated porcine (Sus scrofa) testis cells. Biology of reproduction 76, 43-47, doi:10.1095/biolreprod.106.054999 (2007).
59 Vazquez, J. M. et al. Low-dose insemination in pigs: problems and possibilities. Reprod Domest Anim 43 Suppl 2, 347-354, doi:10.1111/j.1439-0531.2008.01183.x (2008).
60 Umeyama, K. et al. Production of diabetic offspring using cryopreserved epididymal sperm by in vitro fertilization and intrafallopian insemination techniques in transgenic pigs. J Reprod Dev 59, 599-603 (2013).
61 Goel, S. et al. Identification, isolation, and in vitro culture of porcine gonocytes. Biol Reprod 77, 127-137, doi:10.1095/biolreprod.106.056879 (2007).
62 Oatley, M. J., Kaucher, A. V., Yang, Q. E., Waqas, M. S. & Oatley, J. M. Conditions for Long-Term Culture of Cattle Undifferentiated Spermatogonia. Biol Reprod 95, 14, doi:10.1095/biolreprod.116.139832 (2016).
63 Oatley, J. M., Oatley, M. J., Avarbock, M. R., Tobias, J. W. & Brinster, R. L. Colony stimulating factor 1 is an extrinsic stimulator of mouse spermatogonial stem cell self-renewal. Development 136, 1191-1199, doi:10.1242/dev.032243 (2009).
64 Dores, C. & Dobrinski, I. De novo morphogenesis of testis tissue: an improved bioassay to investigate the role of VEGF165 during testis formation. Reproduction 148, 109-117, doi:10.1530/REP-13-0303 (2014).
65 Moreira, F. et al. Leptin and mitogen-activated protein kinase (MAPK) in oocytes of sows and gilts. Anim Reprod Sci 139, 89-94, doi:10.1016/j.anireprosci.2013.03.011 (2013).
66 Cardenas, H. & Pope, W. F. Androgen receptor and follicle-stimulating hormone receptor in the pig ovary during the follicular phase of the estrous cycle. Mol Reprod Dev 62, 92-98, doi:10.1002/mrd.10060 (2002).
67 Young, J. M. et al. Activin B is produced early in antral follicular development and suppresses thecal androgen production. Reproduction 143, 637-650, doi:10.1530/REP-11-0327 (2012).

1. A method of breeding an animal with a genetic edit that causes a failure to thrive phenotype comprising obtaining a host blastocyst, embryo, or morula from the animal with the genetic edit that causes the failure to thrive phenotype and introducing to the host blastocyst, embryo, or morula, a donor cell from a donor animal that comprises a deleted-in-azoospermia like (DAZL) knock out mutation and does not comprise the genetic edit that causes the failure to thrive phenotype to create a chimeric blastocyst, embryo, or morula.

2. The method of claim 1, wherein the failure to thrive phenotype comprises a reduced ability to produce offspring that survive to sexual maturity relative to an animal that does not have the genetic edit that causes the failure to thrive phenotype.

3. The method of claim 1, wherein the failure to thrive phenotype comprises a reduced ability to grow or a reduced ability to reach maturity relative to an animal that does not have the genetic edit that causes the failure to thrive phenotype.

4. The method of claim 1, wherein the failure to thrive phenotype comprises a lineage deficiency phenotype or an organogenesis deficiency phenotype.

5. The method of claim 1, wherein the donor animal does not produce sufficient functional gametes to reproduce.

6. The method of claim 1, wherein the chimeric blastocyst, embryo, or morula is implanted into a surrogate mother to produce an offspring of the animal with the genetic edit that causes the failure to thrive phenotype.

7. The method of claim 6, wherein the offspring comprises the genetic edit that causes the failure to thrive phenotype.

8. The method of claim 7, wherein the offspring is heterozygous for the genetic edit that causes the failure to thrive phenotype.

9. The method of claim 7, wherein the offspring is homozygous for the genetic edit that causes the failure to thrive phenotype.

10. The method of claim 6, wherein the surrogate mother does not comprise the genetic edit that causes the failure to thrive phenotype.

11. The method of claim 7, wherein the offspring does not comprise a genotype of the donor animal.

12. The method of claim 1, wherein the genetic edit that causes the failure to thrive phenotype comprises a genetic edit in a gene selected from the group consisting RNA-Binding Motif Protein 20 (RBM20), Interleukin 2 Receptor Subunit Gamma (IL2Rg), Recombination Activating 2 (RAG2), polycystin-1 (PKD1), polycystin 2 (PKD2), and Fibrocystin/Polyductin (PKHD1).

13. The method of claim 1, wherein the animal with the genetic edit that causes the failure to thrive phenotype or the donor animal or the animal with the genetic edit that causes the failure to thrive phenotype and the donor animal is a livestock animal.

14. The method of claim 13, wherein the livestock animal is selected from the group consisting of cattle, pig, goat, and sheep.

15. The method of claim 14, wherein the pig is a mini-pig.

16. The method of claim 15, wherein the mini-pig is selected from the group consisting of Ossabaw, Goettingen, Yucatan, Bama Xiang Zhu, Wuzhishan and Xi Shuang Banna.

17. The method of claim 1, wherein the donor cell is a stem cell.

18. A chimeric blastocyst, embryo, or morula comprising a host blastocyst, embryo, or morula from an animal with a genetic edit that causes a failure to thrive phenotype and a donor cell from a donor animal with a DAZL knock out mutation and without the genetic edit that causes the failure to thrive phenotype.

19-28. (canceled)

29. A surrogate mother comprising an implanted chimeric blastocyst, embryo, or morula wherein the chimeric blastocyst, embryo, or morula comprises a host blastocyst, embryo, or morula from an animal with a genetic edit that causes a failure to thrive phenotype and a donor cell from a donor animal with a deleted-in-azoospermia like (DAZL) knock out mutation and without the mutation that causes the failure to thrive phenotype.

30-88. (canceled)
